(19) United States
(12) Patent Application Publication
(10) Pub. No.: US 2016/0208008 A1
(43) Pub. Date: Jul. 21, 2016

(54) MONOCLONAL ANTIBODIES TO TRANSFERRIN AND TRANSFERRIN RECEPTOR ANTIGENS, AND USES THEREOF

(71) Applicant: ALPER BIOTECH, LLC, Rockville, MD (US)

(72) Inventor: Özge ALPER, North Bethesda, MD (US)

(21) Appl. No.: 14/855,184

(22) Filed: Sep. 15, 2015

Related U.S. Application Data
(63) Continuation of application No. PCT/US2014/028309, filed on Mar. 14, 2014.
(60) Provisional application No. 61/800,628, filed on Mar. 15, 2013.

(51) Int. Cl. C07K 16/28; A61K 51/10; A61K 39/395; C07K 17/12; A61K 47/48; C12N 7/00; A61K 49/00; C07K 14/705

(52) U.S. Cl. CPC C07K 16/2881; A61K 49/0058; A61K 51/1027; C07K 14/70582; C07K 17/12; A61K 47/48823; C12N 7/00; A61K 39/3955; C07K 2317/565; C07K 2317/56; C07K 2317/24; C07K 2317/41; C07K 2317/76; C07K 2319/30

(57) ABSTRACT

Some embodiments are directed to monoclonal antibodies (mAbs) that bind to transferrin (TF) and transferrin receptor 1 (TFRC), hybridoma lines that secrete these antibodies, and the use of these antibodies to detect TF and TFRC antigens. Some other embodiments are directed to methods and uses for detecting cancer and iron deficiency anemia, as well as methods and uses for distinguishing between early and late stage prostate cancer.

### CROSS-REFERENCE TO RELATED APPLICATIONS

This application claims the benefit of priority to U.S. Provisional Application No. 61/800,628 filed Mar. 15, 2013, the content of which is incorporated by reference in its entirety.

### SEQUENCE LISTING

The instant application contains a Sequence Listing which has been submitted electronically in ASCII format and is hereby incorporated by reference in its entirety. Said ASCII copy, created on Mar. 7, 2014, is named 12112_12_304_SL.txt and is 35,527 bytes in size.

### BACKGROUND & SUMMARY

The present invention relates to monoclonal antibodies (mAbs) and antigen binding fragments thereof that were previously described as binding to transferrin (also referred to herein as “serotransferrin” and “TF”), and have now been further characterized as also binding to transferrin receptor protein 1 (also referred to herein as “transferrin receptor” and “TFRC”). The invention thus encompasses these monoclonal antibodies or antigen binding fragments, and in particular, their uses for detecting TF and TFRC and for diagnosing and treating diseases and conditions related to, or known to be associated with, aberrant TF and TFRC expression and/or activity.

Transferrin (TF) is a glycoprotein with an approximate molecular weight of 76,500 Daltons (76.5 kDa). The function of TF is to transport iron from the intestine, reticuloendothelial system, and liver parenchymal cells to all proliferating cells in the body. TF may also have a physiologic role as granulocyte/pollen-binding protein (GPBP) involved in the removal of certain organic matter and allergens from serum, and may have a further role in stimulating cell proliferation. Human TF is described in the database UniProtKB/Swiss-Prot as TRFE_HUMAN, P02787-1.

Transferrin receptor protein 1 (TFRC) is a glycoprotein with an approximate molecular weight of 85,000 daltons (85 kDa). Cellular uptake of iron occurs via receptor-mediated endocytosis of ligand-occupied TFRC into specialized endosomes. Subsequent acidification of those endosomes leads to iron release. The primary ligand for TFRC is TF, though a second ligand, the hereditary hemochromatosis protein, HFE, competes for binding with TF. TFRC is necessary for development of erythrocytes and the nervous system. Human TFRC is described in the database UniProtKB/Swiss-Prot as TFR1_HUMAN, P02786.

The National Institute of Health (NIH)'s Basic Local Alignment Search Tool (BLAST) indicates that two sets of amino acid sequences in TF are homologous to two sets of amino acid sequences in TFRC. Amino acids 454 to 468 of TF (SASDLTWDNLKGKKS; SEQ ID. NO. 28) share 53% identity with amino acids 118 to 132 of TFRC (AARRLYWDDLKRKLS; SEQ ID NO. 29). Amino acids 483 to 592 of TF (LDGTRKPVEE; SEQ ID NO. 30) share 60% identity with amino acids 101 to 110 of TFRC (LAGTESPVRE; SEQ ID NO. 31). Thus, the cross-reactivity of the mAb described herein is possible, for example, by binding to an epitope shared by TF and TFRC, such as, for example, an epitope related to these homologous sequences.

Biomarkers are molecules that allow for the detection and isolation of a particular protein or cell type, and are typically markers for specific disease states. For example, in prostate cancer, prostate-specific antigen (PSA) is a known biomarker. PSA is known to be present in small quantities in the serum of men with healthy prostates and is often elevated in the serum of men with prostate cancer. In the United States, the U.S. Food and Drug Administration has approved the PSA test for annual screening of prostate cancer in men 50 years and older. However, a 2012 review commissioned by the U.S. Preventative Services Task Force concluded that PSA-based screening result in a small or no reduction in prostate cancer-specific mortality. Moreover, frequent over diagnosis of prostate cancer is associated with the PSA test, resulting in anxiety for receiving false positives, biopsy pain, and other complications from biopsy. Similar issues with biomarker screening have been associated with other cancers, such as the CA-125 test for ovarian cancer. For these reasons, there remains a need to identify new cancer biomarkers that more accurately diagnose patients suffering from particular types of cancer.

TFRC expression has been associated with proliferation of tumor cells. See Sutherland et al. (1981) Proc. Nat'l. Acad. Sci. USA 78(7): 4515-19; I. S. Towbridge & M. B. Omary (1981) Proc. Nat'l. Acad. Sci. USA 78(5): 3039-43; M. E. Bramwell & H. Harris (1978) Proc. R. Soc. London Ser. B. 201: 87-106; M. E. Bramwell & H. Harris (1979) Proc. R. Soc. London Ser. B. 203: 93-99. TFRC overexpression has been observed in a number of cancers, including cancer of the bladder, brain, and breast. See G. J. Seymour et al. (1987) Urol. Res. 15: 341-44; I. Basar et al. (1991) Br. J. Urol. 67(2): 165-68; L. Recht et al. (1990) J. Neurosurg. 72(6): 941-45; J. E. Shindleman et al. (1981) Int. J. Cancer 27(3): 329-34. These observations strongly suggest that TFRC expression could be putative biomarker of malignancy.

Furthermore, it has been shown that brain capillary endothelial cells have a high density of TRFC on their cell surface. See Jeffries et al. (1984) Nature 312: 167-168. Brain capillary endothelial cells constitute the blood brain barrier. See Goldstein et al. (1986) Scientific American 255: 74-83; Padridge, W. M. (1986) Endocrin. Rev. 7:314-339. The blood brain barrier functions to control the environment of the brain by isolating the brain from the blood stream. Id. As a result, delivery of potentially useful therapeutic agents to the brain is extremely challenging. Id. The high expression of TRFC on the cell surface of brain endothelial cells could allow for targeting of TRFC to initiate receptor-mediated delivery of therapeutic or diagnostic agents across the blood brain barrier into the brain. Thus, anti-TFRC antibodies can be utilized in the diagnosis and/or treatment of neurological pathologies.

The invention is based in part on the discovery that a monoclonal antibody specific for TF and TFRC can detect TF and TFRC in tissue, cells, whole blood, serum, plasma, and urine from healthy controls and human subjects suffering from a TF- and/or TFRC-related disorder, such as cancer, a neurological disease or iron deficiency anemia. The inventor's experiments with Alper-TF mAb unexpectedly demonstrate that the antigen recognized by Alper-TF mAb is elevated in patients with cancer. The inventor's experiments with Alper-TF mAb unexpectedly demonstrate that the antigen recognized by Alper-TF mAb is elevated in patients with prostate cancer. Alper-TF mAb can be utilized in immunocytochemical assays, including but not limited to immunohistochemical (IHC) or immunofluorescence (IF) assays, to determine the localization of TF and TFRC, and to determine the severity or stage of cancer depending on its localization and/or expression level. Using the novel antibody of the present invention, the inventor has surprisingly discovered that early-stage and late-stage cancers can be distinguished based upon the staining pattern of the antibody in IHC and IF experiments, as well as based upon the expression level of the antigen in plasma.

### BRIEF DESCRIPTION OF THE DRAWINGS

FIGS. 1A, 1B, AND 1C: FIG. 1A is a Commassie Blue staining of a gel showing a single band at about 77 kDa. FIG. 1B shows one representative image of the results of a Western Blot analysis of purified TF using Alper-TF mAb. FIG. 1C shows another representative image of the results of a Western Blot of purified TF using Alper-TF mAb.

FIGS. 2A, 2B, 2C, 2D, 2E: FIG. 2A depicts the ions score of a Mascot protein database search conducted using mass spectrometry data generated from the antigen bound by Alper-TF mAb in graphical form, where a score >34 indicates identity or extensive homology. FIGS. 2B-2E show the details of the 58 matches identified in this analysis. FIGS. 2B-2E disclose SEQ ID NOS 32-33, 33-34, 34-37, 37-39, 39-40, 40-41, 41-42, 42-43, 43, 43, 43-44, 44-46, 46-48, 48-49, 49-50, 50-51, 51-52, 52-53, 53-56, 56, 56, 56, 56-57, 57, 57, 57, 57-58, 58, 58, 58-59 and 59, respectively, in order of appearance.

FIGS. 3A AND 3B: FIG. 3A shows the optical density (OD) values of TF and/or TFRC levels in healthy and prostate cancer patients as determined by ELISA. FIG. 3B shows the linear correlation between the concentration of purified TF protein and absorbance values (OD), used as a standard curve in this assay.

FIGS. 4A AND 4B: FIG. 4A shows one representative image of the results of an indirect-immunofluorescent staining assay using the Alper-TF mAb with normal prostate cell line OPCN1. FIG. 4B shows one representative image of the results of an indirect-immunofluorescent staining assay using Alper-TF mAb with early-stage prostate cancer cell line OPCT1.

FIGS. 5A, 5B, 5C, AND 5D: FIGS. 5A and 5B show two representative images of the results of an indirect-immunofluorescent staining assay using the Alper-TF mAb with normal prostate cell lines OPCN1 and OPCN2, respectively. FIG. 5C shows two representative image of the results of an Indirect-immunofluorescent staining assay using the Alper-TF mAb with early-stage prostate cancer cell line OPCT1 cells. FIG. 5D shows one representative image of the results of an indirect-immunofluorescent staining assay using the Alper-TF mAb with late-stage prostate cancer cell line LNCaP.

FIG. 6 shows the amino acid sequences of the potential epitopes bound by Alper-TF mAb (SEQ ID NOs: 9-25).

FIG. 7 shows the nucleotide sequence of Alper-TF mAb heavy chain (nucleotides 4-368 of SEQ ID NO: 26).

FIG. 8 shows the nucleotide sequence of Alper-TF mAb light chain (SEQ ID NO: 27).

FIGS. 9A, 9B, 9C, 9D, 9E, 9F, AND 9G: 9A shows the summary of a BLAST analysis of Alper-TF mAb heavy chain. FIGS. 9B-G show, in the top line, the amino acid sequences of Alper-TF mAb heavy chain (SEQ ID NO: 1) and the heavy chain CDR1, CDR2, and CDR3 (SEQ ID NOs: 2, 3 and 4, respectively). The nucleotide sequence is provided in the second line (SEQ ID NO: 26). Amino acid residues are numbered using the convention of Kabat et al., (1991) Sequences of Proteins of Immunological Interest, 5^{th} Edition, Department of Health and Human Services, Public Health Service, National Institutes of Health, Bethesda (NIH Publication No. 91-3242). FIGS. 9B-G also discloses SEQ ID NOS 60-75, respectively, in order of appearance.

FIG. 10A, 10B, 10C, 10D, 10E, AND 10F: FIG. 10A shows the summary of a BLAST analysis of Alper-TF mAb light chain. FIGS. 10B-10F show, in the top line, the amino acid sequences of Alper-TF mAb light chain (SEQ ID NO: 5) and the light chain CDR1, CDR2, and CDR3 (SEQ ID NOs: 6, 7 and 8, respectively). The nucleotide sequence is provided in the second line (SEQ ID NO: 27). Amino acid residues are numbered using the convention of Kabat et al. FIGS. 10B-10F also discloses SEQ ID NOS 76-88, respectively, in order of appearance.

FIGS. 11A AND 11B: FIG. 11A shows a representative image of the results of a direct immunofluorescence assay for Texas Red conjugated-TF (TxR-TF). As expected, TxR-TF, a known endosomal marker, is incorporated into the endosomes during a 10-minute incubation, as demonstrated by the punctate staining in FIG. 11A and FIG. 11B. FIG. 11B shows a representative image of the results of an Indirect immunofluorescence assay for FITC-labeled Alper-TF mAb. Alper-TF mAb fluorescence co-localized with all TxR-TF fluorescence in a similar punctate manner.

FIG. 12 shows one representative image of the results of a Western Blot of recombinant TFRC protein, recombinant TFR II protein (see, UniProt Q9UP52 “TFR2_Human”), and recombinant TF protein using Alper-TF mAb. Alper-TF mAb binds to both recombinant TFRC and TF, but not to TFR II.

FIG. 13 shows the optical density (OD) values of TF and/or TFRC levels in healthy and prostate cancer patients as determined by ELISA. In this assay, the level of antigen bound by Alper-TF mAb in 1 microliter of plasma from normal/healthy patients (n=15), low stage (stage I and II; n=9), and late stage (stage III and IV; n=4) prostate cancer patients are analyzed by competitive ELISA. The results show that Alper-TF mAb can be used to detect both low stage (Stage I and Stage II) and late stage (Stage III and Stage IV) prostate cancers using very low volumes of plasma from human patients. The results also show that Alper-TF mAb can be used to distinguish between low stage (Stage I and Stage II) and late stage (Stage III and Stage IV) prostate cancers using very low volumes of plasma from human patients (higher levels of antigen in low stage prostate cancer). p<0.001 as compared to normal controls.

### BRIEF DESCRIPTION OF CERTAIN SEQUENCES

SEQ ID NO: 1 shows the amino acid sequence of an Alper-TF mAb heavy chain.

SEQ ID NO: 2 shows CDR1 of an Alper-TF mAb heavy chain.

SEQ ID NO: 3 shows CDR2 of an Alper-TF mAb heavy chain.

SEQ ID NO: 4 shows CDR3 of an Alper-TF mAb heavy chain.

SEQ ID NO: 5 shows the amino acid sequence of Alper-TF mAb light chain.

SEQ ID NO: 6 shows CDR1 of an Alper-TF mAb light chain.

SEQ ID NO: 7 shows CDR2 of an Alper-TF mAb light chain.

SEQ ID NO: 8 shows CDR3 of an Alper-TF mAb light chain.

SEQ ID NOs: 9-25 show the amino acid sequence of potential TF and TFRC epitopes.

SEQ ID NO: 26 shows the nucleic acid sequence of an Alper-TF mAb heavy chain.

SEQ ID NO: 27 shows the nucleic acid sequence of an Alper-TF mAb light chain.

SEQ ID NO: 28=SASDLTWDNLKGKKS (amino acids 454 to 468 of TF).

SEQ ID NO: 29=AARRLYWDDLKRKLS (amino acids 118 to 132 of TFRC).

SEQ ID NO: 30=LDGTRKPVEE (amino acids 483 to 592 of TF).

SEQ ID NO: 31=LAGTESPVRE (amino acids 101 to 110 of TFRC).

### DESCRIPTION OF EMBODIMENTS

The present invention provides an antibody or antigen binding fragment capable of binding to a mature or precursor form of TF and TFRC. In one aspect, the present invention includes an antibody or antigen binding fragment thereof that binds to a TF antigen that is a 698 amino acid precursor protein. In one aspect, the present invention includes an antibody or antigen binding fragment thereof that binds to a TF antigen that is a mature TF protein.

In one aspect, the present invention provides an antibody or antigen binding fragment thereof that binds to a TFRC antigen. In certain embodiments, the antibody or antigen binding fragment preferentially binds to a precursor form of TFRC. In another aspect, the present invention provides an antibody or antigen binding fragment capable of binding to a mature form of TFRC.

In certain embodiments, the antibody or antigen binding fragment binds to TF and TFRC with a specific affinity of between 10⁻⁸ M and 10⁻¹¹ M. In other aspects, the present invention provides an antibody capable of binding to a TF or TFRC antigen having post-translational modifications such as glycosylation or phosphorylation. In another aspect, the present invention provides an antibody or antigen binding fragment capable of binding to a mature form of TF or TFRC with post-translational modifications such as glycosylation or phosphorylation. In another embodiment, the antibody or antigen binding fragment may preferentially bind to a mature form of TF or TFRC with a specific affinity of between 10⁻⁸ M and 10⁻¹¹ M.

The present invention provides an antibody or antigen binding fragment capable of selectively modulating the activity of a TF or TFRC antigen (e.g., in a sample or cell). In some embodiments, the antibody or antigen binding fragment is capable of selectively reducing the activity of a precursor TF or TFRC.

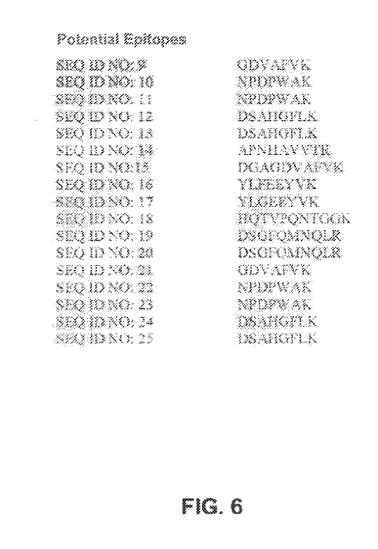

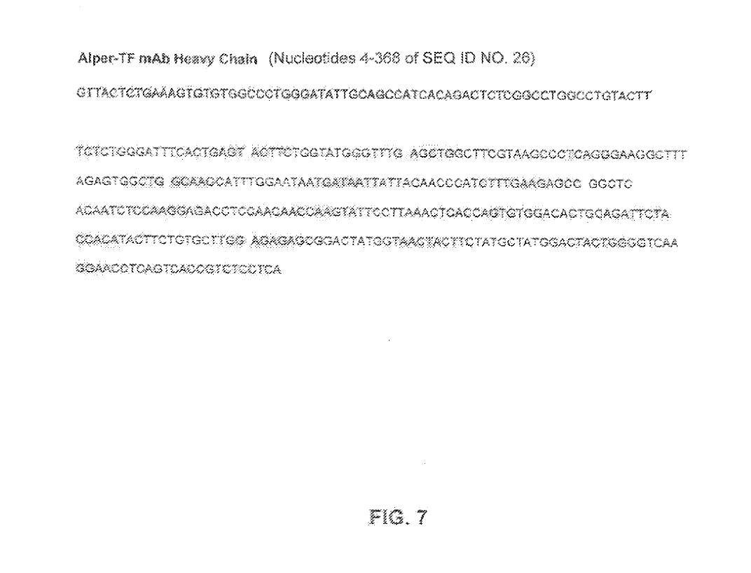

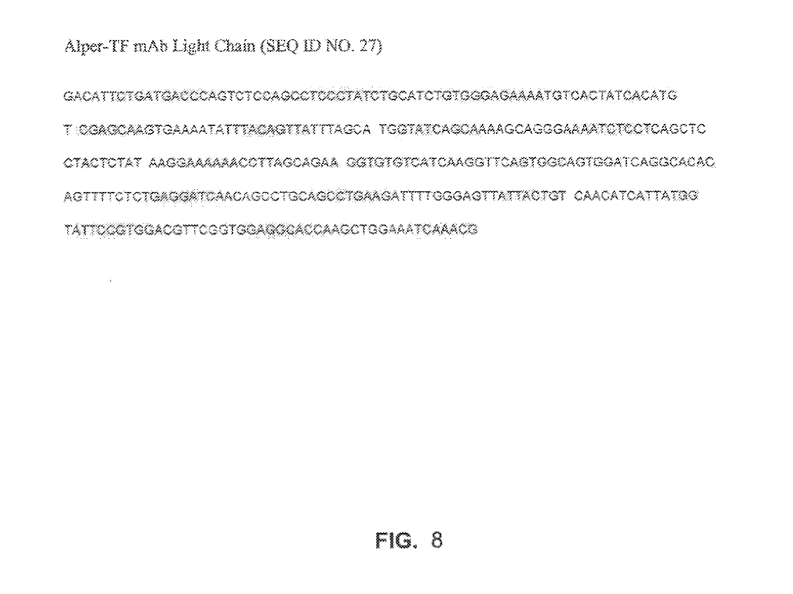

In yet other aspects, the present invention provides an antibody or antigen binding fragment capable of binding to a TF or TFRC epitope consisting of any one of SEQ ID NOs. 9-25, as shown in FIG. 6, or the amino acids shown in SEQ ID NOs. 28-31. In certain aspects, the present invention provides an antibody or antigen binding fragment capable of preferentially binding to a precursor form of TF compared to a mature form of TF. In certain aspects, the present invention provides an antibody or antigen binding fragment capable of preferentially binding to a precursor form of TFRC compared to a mature form of TFRC.

The present invention provides an antibody or antigen binding fragment specific for TF and TFRC, wherein the antibody or antigen binding fragment comprises one or more of the heavy chain complementarity determining region (CDR) antigen binding site sequences set forth in SEQ ID NOs. 2-4, and one or more of the light chain CDR antigen binding site sequences set forth in SEQ ID NOs. 6-8. The antibody specific for TF and TFRC may comprise all three heavy chain CDR antigen binding site sequences CDR1, CDR2, and CDR3 as set forth in SEQ ID NOs. 2-4, and all three light chain CDR antigen binding site sequences CDR1, CDR2, and CDR3 as set forth in SEQ ID NOs. 6-8.

Contemplated is an antibody or antigen binding fragment that binds to TF and TFRC comprising a heavy chain variable domain comprising three CDRs comprising the amino acid sequences of SEQ ID NO: 2, SEQ ID NO: 3, and SEQ ID NO: 4, and a light chain variable domain comprising three CDRs comprising the amino acid sequences of SEQ ID NO: 6, SEQ ID NO: 7, and SEQ ID NO: 8.

In some embodiments, the invention comprises an antibody or antigen binding fragment that binds to human TF or TFRC, wherein the antibody or antigen binding fragment binds to the same epitope as an antibody having a heavy chain comprising the sequence given in SEQ ID NO: 1 and a light chain comprising the sequence given in SEQ ID NO: 5.

In other aspects, the present invention provides an isolated DNA sequence which encodes the heavy chain of an antibody, wherein the antibody has specificity for TF and TFRC, and wherein the variable domain of said heavy chain comprises at least one CDR selected from the heavy chain CDRs of CDR1, CDR2, and CDR3 set forth in SEQ ID NOs. 2-4.

In other aspects, the present invention provides an isolated DNA sequence which encodes the light chain of an antibody, wherein the antibody has specificity for TF and TFRC, and wherein the variable domain of said light chain comprises at least one CDR selected from the light chain CDRs of CDR1, CDR2, and CDR3 set forth in SEQ ID NOs. 6-8.

In one embodiment, the isolated DNA sequence comprises DNA encoding the amino acids of all three CDRs from the heavy chain and all three CDRs from the light chain.

In yet other aspects, the present invention provides a method of characterizing TF and TFRC expression by cells in a biological sample by (a) obtaining said sample; (b) contacting said sample with an antibody or antigen binding fragment capable of preferentially detecting TF and TFRC; and (c) determining quantity and/or localization of said TF and TFRC. Detection of TF and/or TFRC indicates that the sample expresses TF or TFRC. Detection is also indicative of cancer. In some embodiments, detection of TF and/or TFRC indicates that the sample is from a human having prostate cancer.

In yet other aspects, the present invention provides an immunoassay for detecting TF and TFRC in a biological sample. The immunoassay may comprise: (a) contacting a biological sample with an antibody described herein; and (b) qualitatively or quantitatively determining the formation of an immune complex of the antibody and TF and/or TFRC. In some embodiments, the immunoassay is an ELISA. In various embodiments, the immunoassay is a sandwich ELISA. In some embodiments, the immunoassay is a circulating tumor cell assay.

In yet other aspects, the present invention provides an immunoassay for detecting an antigen bound by an Alper-TF mAb, in a biological sample. The immunoassay may comprise: (a) contacting a biological sample with an Alper-TF mAb: and (b) qualitatively or quantitatively determining the formation of an immune complex of the antibody and TF and/or TFRC. In some embodiments, the immunoassay is an ELISA. In various embodiments, the immunoassay is a sandwich ELISA. In some embodiments, the immunoassay is a circulating tumor cell assay.

In yet other aspects, the immunoassay is an immunocytochemical assay, including but not limited to an immunohistochemical or immunofluorescence assay. The immunocytochemical (ICC) assay may be performed on tissue, cells, whole blood, plasma, serum, or urine. The ICC assay may be used to detect TF or TFRC. An ICC method is contemplated wherein a biological sample from a patient diagnosed with cancer or in need of diagnosis is contacted with an antibody or antigen binding fragment described herein; and the formation of immune complex of the antibody and TF or the antibody and TFRC is qualitatively or quantitatively determined.

The level and localization of TF or TFRC can provide a diagnosis of cancer when compared to a healthy non-cancerous control or when compared to an earlier sample from the same patient. As described herein, the antigen recognized by Alper-TF mAb is increased in cancer. As described herein, the antigen recognized by Alper-TF mAb is increased in prostate cancer. Moreover, the localization of the antigen recognized by Alper-TF mAb to the endosomes (punctate cytoplasmic staining), as well as localization to the cytoplasm in the absence of punctate staining, indicates a diagnosis of cancer. Early stage cancer can be detected and diagnosed by localization of the antigen recognized by Alper-TF mAb to endosomes (punctate cytoplasmic staining). Late stage cancer can be detected and diagnosed by localization of the antigen recognized by Alper-TF mAb to the cytoplasm in the absence of punctate/endosomal staining.

In each method and use described herein, the biological sample may be selected from tissue, cells, whole blood, serum, plasma, and urine. The biological sample may be selected from a human subject diagnosed with cancer or from a human subject in need of diagnosis of cancer. In some embodiments, the cancer is prostate cancer.

In other aspects, the present invention provides a method of characterizing TF expression by cells in a sample comprising: (a) obtaining a sample from a subject; (b) contacting the sample with an antibody or antigen binding fragment capable of preferentially detecting TF antigen; and (c) determining the quantity or localization of the antigen.

In yet other aspects, the present invention provides a method of characterizing TFRC expression by cells in a sample comprising: (a) obtaining a sample from a subject; (b) contacting the sample with an antibody or antigen binding fragment capable of preferentially detecting a TFRC antigen; and (c) determining the quantity or localization of the antigen.

In some aspects, the present invention relates to a method of treating cancer comprising administering an effective amount of a composition comprising an antibody or antigen binding fragment capable of detecting a TFRC antigen.

In some aspects, the present invention relates to a method of treating iron deficiency anemia comprising administering an effective amount of a composition comprising an antibody or antigen binding fragment capable of detecting a TFRC antigen.

In another aspect, the present invention relates to a method for delivering a neuropharmaceutical or neurodiagnostic agent across the blood brain carrier to the brain of a subject comprising administering an effective amount of a composition comprising an antibody or antigen binding fragment capable of detecting a TFRC antigen

### DEFINITIONS

Antibody: This refers to single chain, two-chain, and multi-chain proteins and glycoproteins belonging to the classes of polyclonal, monoclonal, chimeric, and hetero immunoglobulins; it also includes synthetic and genetically engineered variants of these immunoglobulins. “Antibody binding fragment” or “antibody fragment” includes Fab, Fab′, F(ab′)₂, and Fv fragments, as well as any portion of an antibody having specificity toward a desired target epitope or epitopes.

Monoclonal Antibody: This refers to antibodies that are identical because they are produced by one type of immune cell that are all clones of a single parent cell. The monoclonal antibodies of the present invention can include intact monoclonal antibodies, antibody fragments, conjugates, or fusion proteins, which contain a V_{H} and a V_{L} where the CDRs form the antigen binding site.

Chimeric Antibody: This refers to an antibody which includes sequences derived from two different antibodies, which typically are of different species. Most typically, chimeric antibodies include human and non-human antibody fragments, generally human constant and non-human variable regions. Humanized antibodies can or cannot be considered chimeric.

Humanized Antibody: This refers to an antibody derived from a non-human antibody. The humanized antibody retains or substantially retains the antigen-binding properties of the parent antibody but is less immunogenic in humans than its parent antibody.

Antibody Conjugates, Fusion Proteins, and Bispecific Antibodies: These refer to monoclonal antibodies conjugated by chemical or non-chemical methods with radionuclides, drugs, macromolecules, or other agents.

Alper-TF mAb: This term refers to an antibody comprising a heavy chain variable domain comprising at least one CDR selected from the group consisting of: the amino acid sequence of SEQ ID NO: 2, the amino acid sequence SEQ ID NO: 3, and the amino acid sequence SEQ ID NO: 4, and a light chain variable domain comprising at least one CDR selected from the group consisting of: the amino acid sequence of SEQ ID NO: 6, the amino acid sequence of SEQ ID NO: 7, and the amino acid sequence of SEQ ID NO: 8.

Antigen; This refers to one or more molecules or one or more portions of a molecule capable of being bound by an antibody which is additionally capable of inducing an animal to produce an antibody capable of binding to an epitope of that antigen. An antigen can have one or more than one epitope. The specific reaction referred to above is meant to indicate that the antigen will react, in a highly preferential manner, with its corresponding antibody and not with the multitude of other antibodies which can be evoked by other antigens. The binding of antigen to antibody must be above background levels.

Epitope: This refers to that portion of any molecule capable of being recognized by, and bound by, an antibody. In general, epitopes consist of chemically active surface groupings of molecules, for example, amino acids or sugar side chains, and have specific three-dimensional structural characteristics as well as specific charge characteristics. The epitopes of interest for the present invention are epitopes comprising amino acids, and are shown in FIG. 6.

Complementarity Determining Region, or CDR: This refers to amino acid sequences which together define the binding affinity and specificity of the natural Fv region of a native immunoglobulin binding site. The light and heavy chains of an immunoglobulin each have three CDRs. The numbering convention delineated by Kabat et al., (1991) Sequences of Proteins of Immunological Interest, 5th Edition, Department of Health and Human Services, Public Health Service, National Institutes of Health, Bethesda (NIH Publication No. 91-3242) is used where no other numbering is provided.

Framework Region or FWR: This refers to amino acid sequences interposed between CDRs. These portions of the antibody serve to hold the CDRs in an appropriate orientation for antigen binding.

Specificity Determining Residue or SDR: This refers to amino acid residues that are unique to Alper-TF mAb when compared to other IgGs. Preferentially, the SDR is the part of an immunoglobulin that is directly involved in antigen contact. The sequence of the CDRs may be altered at any residue except those indicated as an SDR.

Constant Region: This refers to the portion of an antibody molecule which confers effector functions. A heavy chain constant region can be selected from any of five isotypes: alpha, delta, epsilon, gamma, or mu. Heavy chains of various subclasses (such as the IgG subclass of heavy chains) are responsible for different effector functions. Thus, by choosing the desired heavy chain constant region, humanized antibodies with the desired effector function can be produced. A light chain constant region can be of the kappa or lambda type, preferably the kappa type.

Immunogenicity: A measure of the ability of an antigen to elicit an immune response (humoral or cellular) when administered to a recipient. The present invention is concerned with the immunogenicity of antibodies to TF and antibodies to TFRC.

Immunoreactivity: A measure of the ability of an immunoglobulin to recognize and bind to a specific antigen.

TF Antibodies or TF mAbs: These terms refer to antibodies that bind to TF or a TF epitope, or proteins that are specifically bound by the same protein as a protein with the epitope for Alper-TF mAb as shown in FIG. 6 (SEQ ID NO: 9-25), which may be a modified or precursor form of the protein that is produced by cancer cells. The antibodies include variants, such as chimeric, humanized, and other variants known to those skilled in the art. TF antibodies are said to be specific for a TF antigen if they exhibit preferential binding to the same TF antigen as bound by Alper-TF mAb at least 85% of the time, at least 90% of the time, or, in a preferred aspect, at least 95% of the time relative to background staining.

TFRC Antibodies or TFRC mAbs: These terms refer to antibodies that bind to TFRC or a TFRC epitope, and bind to proteins that are specifically bound by the same protein as a protein with the epitope for Alper-TF mAb as shown in FIG. 6 (SEQ ID NO: 9-25), which may be a modified or precursor form of the protein that is produced by cancer cells. The antibodies include variants, such as chimeric, humanized, and other variants known to those skilled in the art. TFRC antibodies are said to be specific for a TFRC antigen if they exhibit preferential binding to the same TFRC antigen as bound by Alper-TF mAb at least 85% of the time, at least 90% of the time, or, in a preferred aspect, at least 95% of the time relative to background staining.

TF and TFRC as used herein are TF antigens and TFRC antigens, respectively.

TF antigens and TFRC antigens: These terms refer to expression products bound by Alper-TF mAb, which can be used as antigens, target molecules, biomarkers, or any combination thereof. A TF antigen can be produced by a TF gene and homologues of a TF gene and can include various modifications, precursor forms, mature forms, or secreted forms of TF bound by Alper-TF mAb and produced by a cell expressing that TF antigen, such as a cancer cell. A TFRC antigen can be produced by a TFRC gene and homologues of a TFRC gene and can include various modifications, precursor forms, mature forms, or secreted forms of TFRC bound by Alper-TF mAb and produced by a cell expressing that TFRC antigen, such as a cancer cell.

Substantially Similar Binding Properties: This refers to an antibody, such as a humanized antibody or fragments thereof which retain the ability to preferentially bind an antigen recognized by the parent antibody used to produce the antibody, such as a humanized antibody, or fragments thereof. Preferably, the affinity of a chimeric antibody, humanized antibody, or antibody fragment is at least about 10% of the affinity of the parent antibody, more preferably at least about 25%, even more preferably at least about 50%. Most preferably, a chimeric antibody, preferably a humanized antibody, or antibody fragments thereof exhibit an antigen-binding affinity that is at least about 75% of the affinity of the parent antibody. Methods for assaying antigen-binding affinity are known in the art and include half-maximal binding assays, competition assays, and Scatchard analysis. In a preferred aspect, antigen-binding affinity is determined using a competition assay.

Substantially Homologous: This refers to immunoglobulin sequences that exhibit at least about 85% identity, more preferably about 90% identity, most preferably about 95% identity with a reference immunoglobulin sequence, where % identity is determined by comparing the number identical of amino acid residues between the two immunoglobulins, where the positions of the amino acid residues are indicated using the Kabat numbering scheme.

Substantially pure: For the purpose of the present invention, substantially pure refers to a homogeneous preparation preferably of a TF antibody, TFRC antibody. TF antibody fragment. TFRC antibody fragment, or other chemical or biological agents. Substantially pure immunoglobulins of at least 80% homogeneity are preferred, with about 90% to about 95% homogeneity being more preferred, and 98% to 99% or more homogeneity is most preferred and is generally considered acceptable for pharmaceutical uses.

Immunocytochemistry: As used herein, immunocytochemistry (ICC) refers to assays that use antibodies to detect specific peptide, proteins, protein antigens, or epitopes that are bound by the antibodies. The antibodies may be labeled with a detection agent or non-labeled. Immunofluorescence is a type of immunocytochemistry that utilizes fluorescent detection. Immunohistochemistry (IHC) is a type of immunocytochemistry that specifically analyzes peptides, protein, protein antigens, or epitopes that are bound by the antibodies in sections of biological tissues.

Immunoassay: As used herein, immunoassay refers to a test that measures the presence, amount, or concentration of a molecule using an antibody or antibody fragment. Non-limiting examples of immunoassays include immunohistochemistry, immunofluorescence, enzyme-linked immunosorbent assays (ELISAs), enzyme immunoassays (EIAs), radioimmunoassays (RIAs), flow cytometry, real-time immunoquantitative polymerase chain reactions (iqPCRs), protein microarrays, surface plasmon resonance, and assays for detecting circulating tumor cells.

Alper-TF Antibodies and Alper-TF Antibody Fragments

The present invention provides isolated antibodies that bind to TF and TFRC, including Alper-TF mAb, as well as antibodies and antigen binding fragments thereof that are capable of binding to the same epitope as is bound by Alper-TF mAb. Antibodies or antibody fragments include those that are specific for at least one TF or TFRC form, at least the same TF or TFRC form bound by Alper-TF mAb. In certain embodiments, the antibodies and antibody fragments thereof can be used to detect a precursor and/or mature form of TF or TFRC within tissues, cells, blood, serum, plasma, and urine.

The antibodies and antibody fragments, including Alper-TF mAb, detect an approximately 77 kDa TF antigen and an approximately 85 kDa TFRC antigen. The antibodies and antibody fragments are useful in detecting cancer in tissues, cells, blood, serum, plasma, and urine. The antibodies and antibody fragments are useful in detecting prostate cancer in tissues, cells, blood, serum, plasma, and urine. The antibodies and antibody fragments are useful in detecting circulating tumor cells in blood, serum, plasma, and urine.

Increased levels of TF are detected in cancerous tissues, cells, blood, serum, plasma, and urine, when probed with an anti-TF antibody of the invention, including Alper-TF mAb, and when compared to a non-cancerous control. In one aspect, the TF antigen preferentially bound by Alper-TF mAb is localized in the early endosomes of subjects with early-stage cancer, including prostate cancer. In another aspect, the TF antigen preferentially bound by Alper-TF mAb moves into late endosomes in cells of subjects with later stages of cancer. In one aspect, levels of soluble TF antigen in late endosomes of cancer cells are significantly associated with decreased chance of survival relative to the chance of survival of patients with soluble TF antigen in early endosomes of prostate cancer cells, observed in patients with early-stage prostate cancer.

Similarly, increased levels of TFRC are detected in cancerous tissues, cells, blood, serum, plasma, and urine, when probed with an anti-TFRC antibody of the invention, such as Alper-TF mAb, and when compared to a non-cancerous control. In one aspect, the TFRC antigen preferentially bound by Alper-TF mAb is localized in the early endosomes of subjects with early-stage cancer, including prostate cancer. In another aspect, the TFRC antigen preferentially bound by Alper-TF mAb moves into late endosomes in cells of subjects with later stages of cancer. In one aspect, levels of TFRC antigen in late endosomes of cancer cells are significantly associated with decreased chance of survival relative to the chance of survival of patients with TFRC antigen in early endosomes of prostate cancer cells, observed in patients with early-stage prostate cancer.

In yet another aspect, the TF and TFRC antigen bound by Alper-TF mAb is localized to exosomes. Exosomes are nanometer-sized vesicles secreted by a wide range of mammalian cell types. Exosomes are a notable feature of cancer and malignancy. For example, exosome secretion is increased in cancer cells. Tumor-antigen enrichment of exosomes is also associated with cancer cells. Mitchell et al. identified the utility of measuring PSA in exosomes concentrated from urine, finding that PSA was present in exosomes concentrated from the urine of 20 of 24 prostate cancer specimens but notably absent from healthy donor specimens. Journal of Translational Medicine (2009) 7: 4. One embodiment of the present invention includes Alper-TF antibodies and Alper-TF antibody fragments capable of detecting TF and TFRC antigen in urinary exosomes. The detection of TF and TFRC in urinary exosomes indicates the presence of cancer. Another embodiment of the present invention includes Alper-TF antibodies and Alper-TF antibody fragments capable of detecting TFRC antigen in urinary exosomes. The detection of TFRC in urinary exosomes indicates the presence of cancer.

One embodiment includes TF antibodies and TF antibody fragments capable of binding to the same TF antigen as bound by Alper-TF mAb with a specific affinity of between 10⁻⁸ M and 10⁻¹¹ M. Another embodiment includes a TF antibody or TF antibody fragment capable of selectively modulating the activity of such a TF antigen in a cell. Another embodiment includes a TF antibody or TF antibody fragment capable of selectively reducing the activity of such a TF antigen in a cell.

Yet, another embodiment includes TFRC antibodies and TFRC antibody fragments capable of binding to the same TFRC antigen as bound by Alper-TF mAb with a specific affinity of between 10⁻⁸ M and 10⁻¹¹ M. Another embodiment includes a TFRC antibody or TFRC antibody fragment capable of selectively modulating the activity of such a TFRC antigen in a cell. Another embodiment includes a TFRC antibody or TFRC antibody fragment capable of selectively reducing the activity of such a TFRC antigen in a cell.

A TF antibody, TFRC antibody, TF antibody fragment, or TFRC antibody fragment can be, without limitation, a monoclonal antibody, a chimeric antibody, a humanized antibody, or an antibody conjugate.

A TF antibody, TFRC antibody, TF antibody fragment, or TFRC antibody fragment can be any gamma globulin protein found in blood or other bodily fluids of vertebrates, and used by the host immune system to identify and neutralize foreign objects, such as bacteria and viruses. In another aspect, the antibody or antibody fragment can be selected from an antibody, a monoclonal antibody, a chimeric antibody, a humanized antibody, or an antibody conjugate. In yet another aspect, a TF antibody, TFRC antibody, TF antibody fragment, or TFRC antibody fragment can be any type of immunoglobulin protein, such as IgA, IgD, IgE, IgG or IgM.

In one aspect, a TF antibody or TF antibody fragment is capable of reducing the activity of that bound TF form, including a soluble precursor form. In another aspect, a TF antibody or TF antibody fragment is capable of reducing the activity of TF in a mature form. In yet another aspect, a TFRC antibody or TFRC antibody fragment is capable of reducing the activity of that bound TFRC form, including a soluble precursor form. In another aspect, a TFRC antibody or TFRC antibody fragment is capable of reducing the activity of TFRC in a mature form.

In another aspect of the present invention, a TF antibody or TF antibody fragment is capable of preferentially binding to a mature form of TF protein. In one aspect of the present invention, a TF antibody or TF antibody fragment is capable of preferentially binding to a precursor form of TF protein. In another aspect of the present invention, a TF antibody or TF antibody fragment is capable of binding to a mature or precursor form or forms of a TF antigen. In such aspects, such preferential binding of a TF antigen can be relative to background staining. In a particular aspect, such preferential binding is relative to a mature TF antigen. In another particular aspect, such preferential binding to a TF antigen is relative to a TF that is nuclear bound or membrane associated. In another aspect of the present invention, antibodies or antibody fragments can be used to detect a mature form of TF.

In another aspect of the present invention, a TFRC antibody or TFRC antibody fragment is capable of preferentially binding to a mature form of TFRC protein. In one aspect of the present invention, a TFRC antibody or TFRC antibody fragment is capable of preferentially binding to a precursor form of TFRC protein. In another aspect of the present invention, a TFRC antibody or TFRC antibody fragment is capable of binding to a mature or precursor form or forms of a TFRC antigen. In such aspects, such preferential binding of a TFRC antigen can be relative to background staining. In a particular aspect, such preferential binding is relative to a mature TFRC antigen. In another particular aspect, such preferential binding to a TFRC antigen is relative to a TFRC that is nuclear bound or membrane associated. In another aspect of the present invention, antibodies or antibody fragments can be used to detect a mature form of TFRC.

In an aspect of the present invention, a TF antibody or TF antibody fragment is capable of preferentially binding to TF protein localized to endosomes. In another aspect of the present invention, a TF antibody or TF antibody fragment is capable of preferentially binding to TF protein localized to multivesicular bodies. In yet another aspect of the present invention, a TF antibody or TF antibody fragment is capable of preferentially binding to TF protein localized to exosomes. In such aspects, such preferential binding of a TF antigen can be relative to background staining. In a particular aspect, such preferential binding is relative to TF protein localized to the cytoplasm. In another particular aspect, such preferential binding to a TF antigen is relative to TF protein that is nuclear bound or membrane associated.

In an aspect of the present invention, a TFRC antibody or TFRC antibody fragment is capable of preferentially binding to TFRC protein localized to endosomes. In another aspect of the present invention, a TFRC antibody or TFRC antibody fragment is capable of preferentially binding to TFRC protein localized to multivesicular bodies. In yet another aspect of the present invention, a TFRC antibody or TFRC antibody fragment is capable of preferentially binding to TFRC protein localized to exosomes. In such aspects, such preferential binding of a TFRC antigen can be relative to background staining. In a particular aspect, such preferential binding is relative to TFRC protein localized to the cytoplasm. In another particular aspect, such preferential binding to a TFRC antigen is relative to TFRC protein that is nuclear bound or membrane associated.

In an aspect of the present invention, preferential binding is relative to background staining. In another aspect, the preferential binding is at least 2-fold, 3-fold, 4-fold, 5-fold, 10-fold, 100-fold, 1,000-fold, 10,000-fold or 1,000,000-fold increased relative to control. Methods for assaying antigen-binding affinity are known in the art and include half-maximal binding assays, competition assays, and Scatchard analysis, as set forth in Ausubel et al., Current Protocols in Molecular Biology (John Wiley & Sons Inc.). In a preferred aspect, antigen-binding affinity is assayed using a competition assay.

In an aspect, a TF antibody or TF antibody fragment binds TF or a particular form of TF such as a secreted, precursor form or a secreted, mature form, and/or a form with post-transcriptional processing such as phosphorylation or glycosylation, with a specific affinity of greater than 10⁻⁷ M, 10⁻⁸ M, 10⁻⁹ M, 10⁻¹⁰ M, or 10⁻¹¹ M, or between 10⁻⁸ M-10⁻¹¹ M, 10⁻⁹ M-10⁻¹⁰ M, and 10⁻¹⁰ M-10⁻¹¹ M. In a preferred aspect, specific activity is measured using a competitive binding assay as set forth in Ausubel.

In an aspect, a TFRC antibody or TFRC antibody fragment binds TFRC or a particular form of TFRC such as a secreted, precursor form or a secreted, mature form, and/or a form with post-transcriptional processing such as phosphorylation or glycosylation, with a specific affinity of greater than 10⁻⁷ M, 10⁻⁸ M, 10⁻⁹ M, 10⁻¹⁰ M, or 10⁻¹¹ M, or between 10⁻⁸ M-10⁻¹¹ M, 10⁻⁹ M-10⁻¹⁰ M, and 10⁻¹⁰ M-10⁻¹¹ M. In a preferred aspect, specific activity is measured using a competitive binding assay as set forth in Ausubel.

TF antibodies, TF antibody fragments, TFRC antibodies, and TFRC antibody fragments can optionally be immobilized on a solid phase, detectably labeled, or conjugated to a cytotoxic radionuclide, a cytotoxic drug, or a cytotoxic protein and the like. TF antibodies, TF antibody fragments, TFRC antibodies, and TFRC antibody fragments can optionally be labeled. Labels include, but are not limited to, fluorescent and radioisotope labeling.

TF antibodies, TF antibody fragments, TFRC antibodies, and TFRC antibody fragments of the present invention can detect TF or TFRC in human cells, more preferably human cancer cells, such as cancer cells of human breast, ovary, cervix, prostate, colon, stomach, kidney, liver, head, neck, lung, blood, pancreas, skin, testis, thyroid and brain. Expressed TF or TFRC antigens can include any form of the gene product, although particularly preferred aspects relate to the detection of the soluble or secreted form of TF. Such antigens can also include gene-produced homologues of the TF or TFRC gene and modified TF or TFRC antigens expressed by cancer cells. In one aspect, the modified TF gene product is phosphorylated. In another aspect, the modified TFRC gene product is phosphorylated.

In one aspect, TF antibodies. TF antibody fragments, TFRC antibodies, and TFRC antibody fragments include those capable of binding to the epitopes comprising or consisting of those shown in FIG. 6, such as SEQ ID NOs: 9-25 and 28-31, or fragments of these amino acids. Antibodies or antibody fragments can preferentially be used to detect the TF and TFRC epitopes comprising or consisting of those shown in FIG. 6, such as SEQ ID NOs: 9-25 or fragments of these amino acids. The invention also includes TF antibodies, TF antibody fragments, TFRC antibodies, and TFRC antibody fragments specific to TF and TFRC expression products that contain antigen binding sites that are substantially homologous to proteins comprising or consisting of the amino acids of SEQ ID NOs: 9-25 and 28-31 or that result in substantially similar binding properties. Such antibodies or fragments thereof can be capable of binding to epitopes that are 95%, 90%, 85%, or 80% identical to one or more of the TF or TFRC epitopes comprising or consisting of those shown in FIG. 6, such as SEQ ID NOs: 9-25 and 28-31 or fragments of these amino acids.

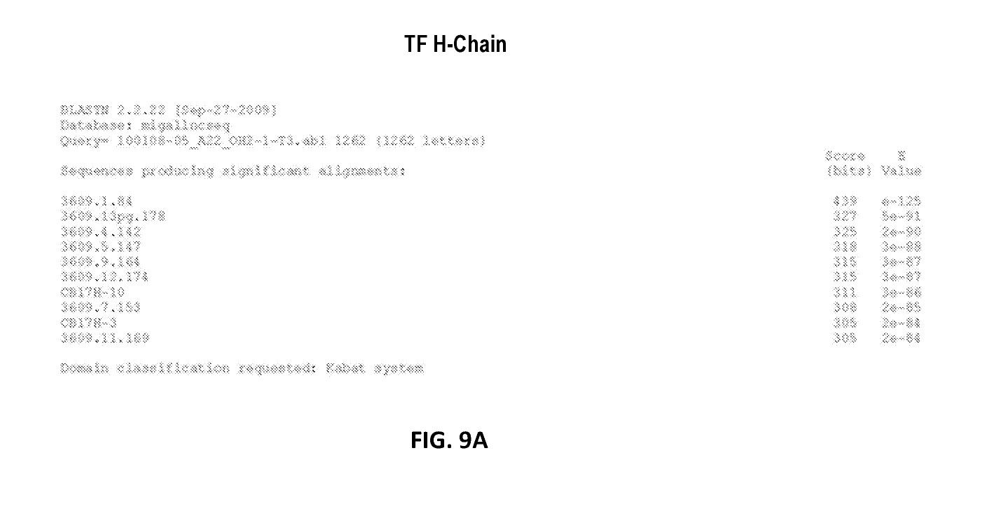

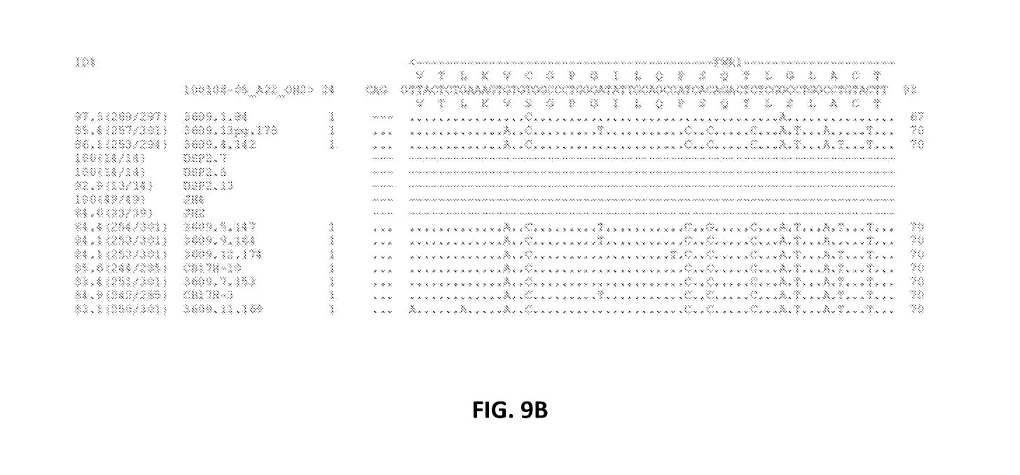

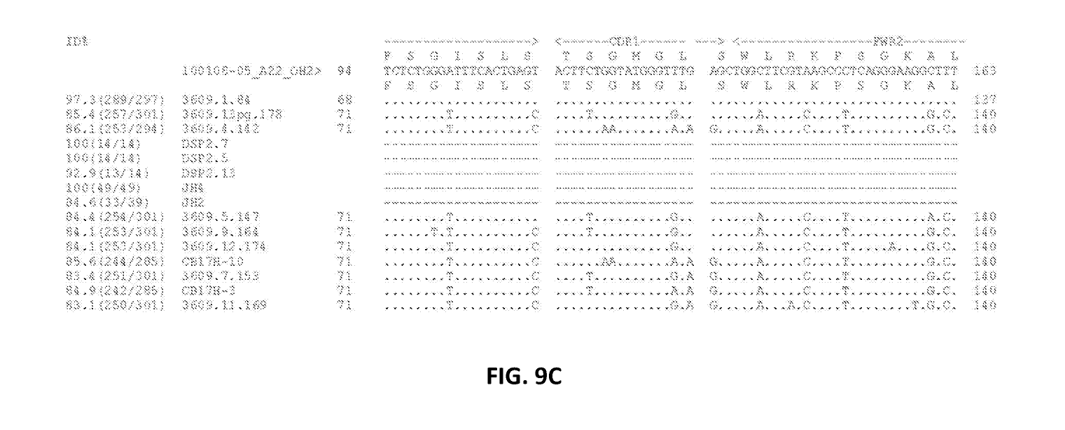

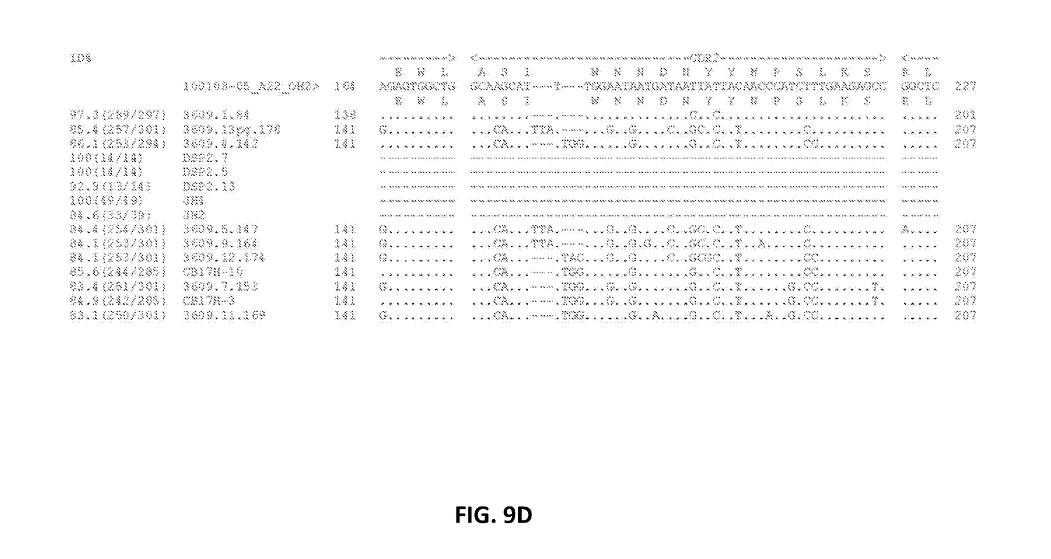

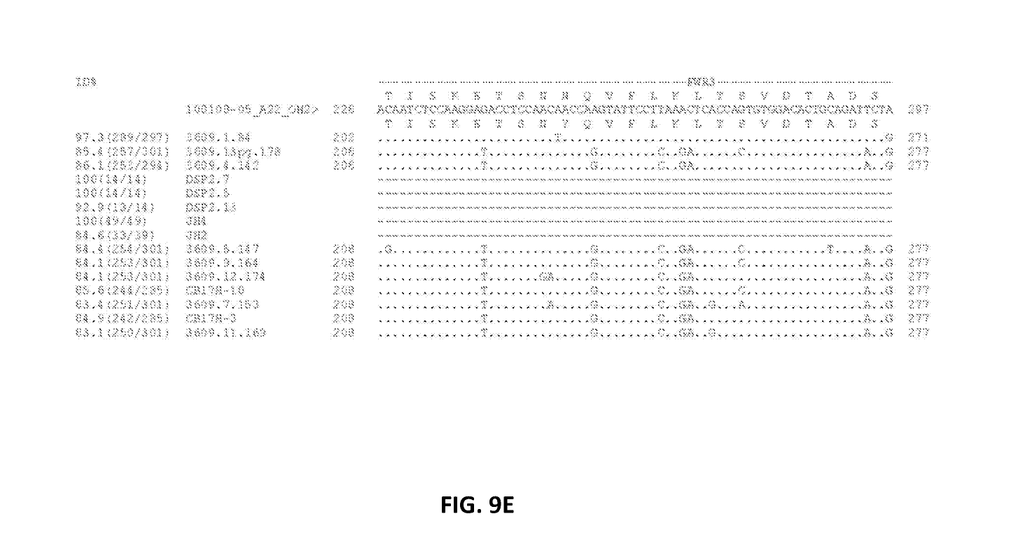

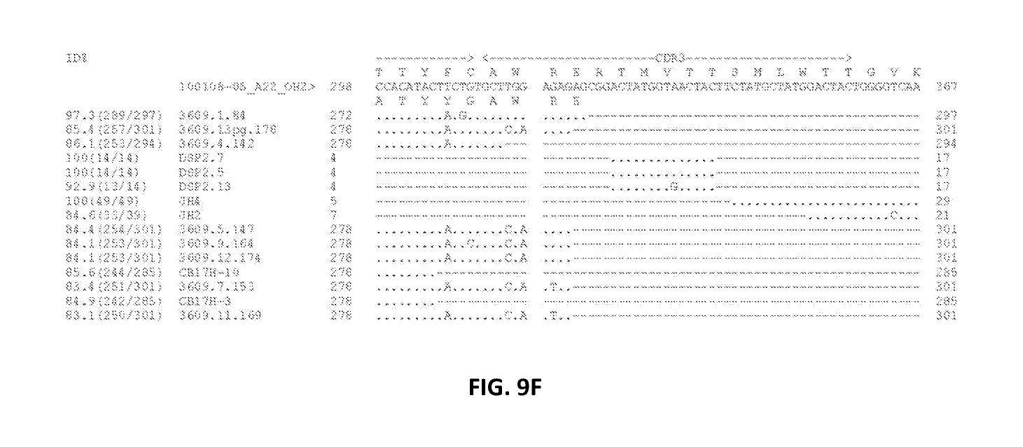

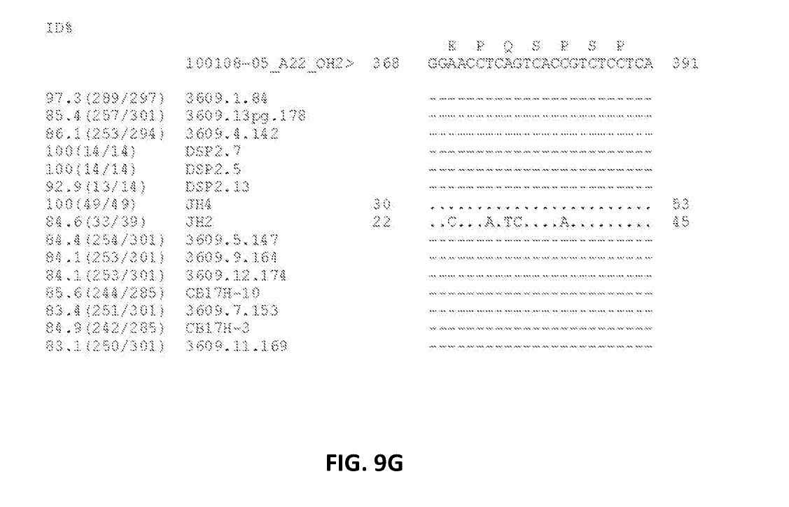

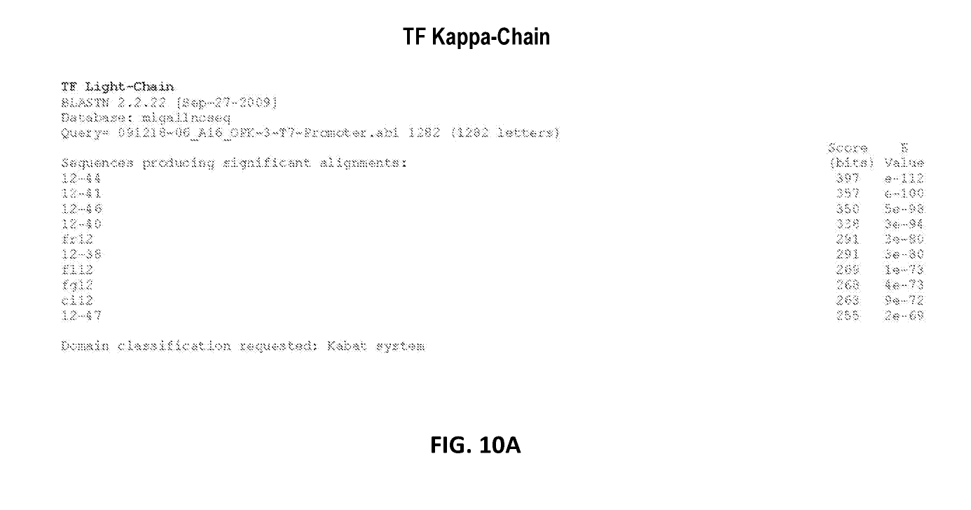

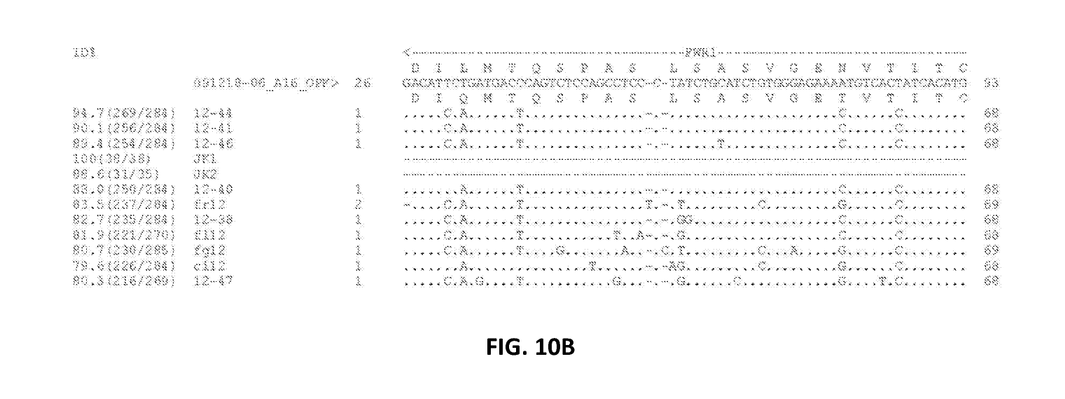

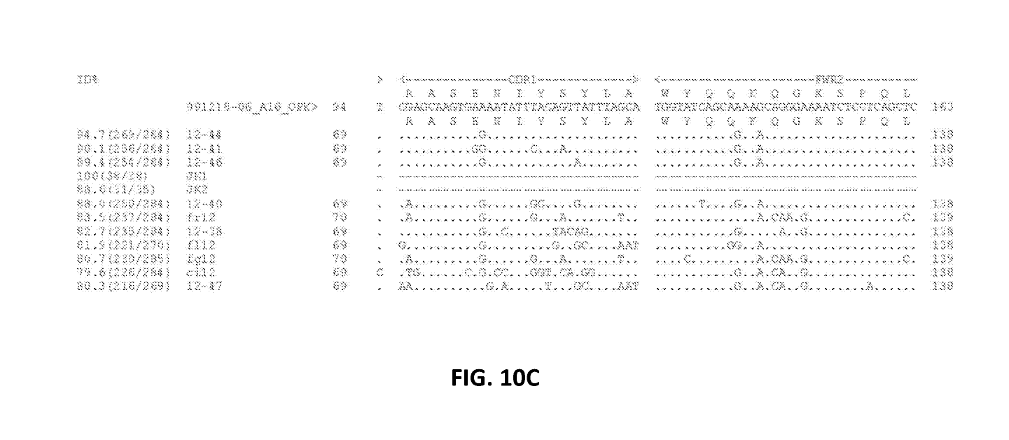

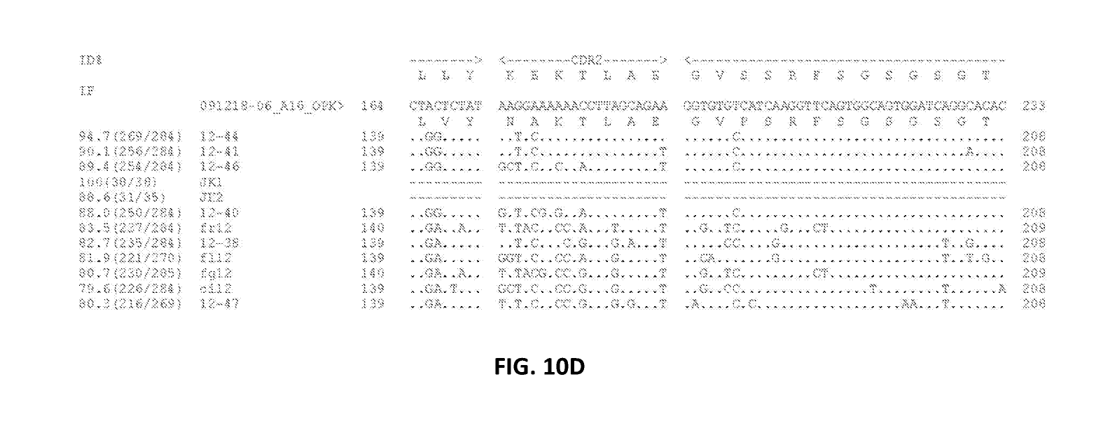

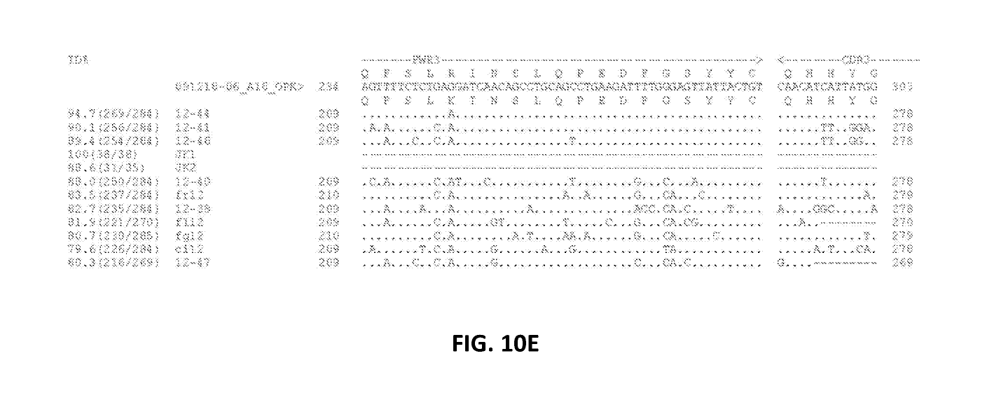

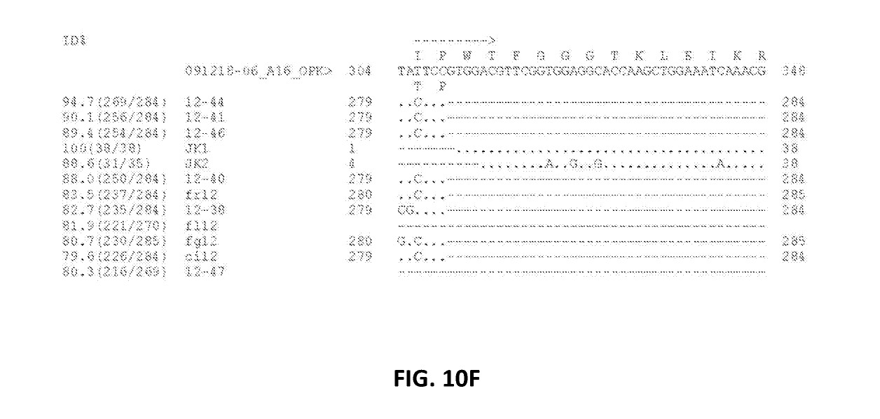

In another aspect, the present invention includes an antibody or an antibody fragment that binds TF and TFRC, wherein the antibody or antibody fragment comprises, consists of, or has, at least one of the heavy chain CDR antigen binding site amino acid sequences CDR1, CDR2, and CDR3 (SEQ ID NOs: 2, 3, and 4, respectively, as set forth in FIG. 9), and/or at least one of the light chain CDR antigen binding site amino acid sequences CDR1, CDR2 and CDR3 (SEQ ID NOs.: 6, 7, and 8, respectively, as set forth in FIG. 10). A TF antibody, TF antibody fragment, TFRC antibody, or TFRC antibody fragment may include any single CDR shown in FIGS. 9 and 10, alone or in combination. By way of example, a TF antibody, TF antibody fragment, TFRC antibody, or TFRC antibody fragment may include CDR1 and CDR2 from both heavy and light chains of FIGS. 9 and 10 (SEQ ID NOs.: 2, 3, 6, and 7, respectively). In other embodiments, a TF antibody, TF antibody fragment, TFRC antibody, or TFRC antibody fragment may include CDR1, CDR2, CDR3 from both heavy and light chains of FIGS. 9 and 10 (SEQ ID NOs.: 2, 3, 4, 6, 7, and 8, respectively). In yet other embodiments, a TF antibody, TF antibody fragment, TFRC antibody, or TFRC antibody fragment may include the full heavy and light chain amino acid sequences illustrated in FIGS. 9 and 10 (SEQ ID NOs.: 1, 26 and 5, 27).

The invention also includes TF antibodies, TF antibody fragments, TFRC antibodies, and TFRC antibody fragments specific to TF and TFRC expression products that contain antigen binding sites that are substantially homologous to these or that result in substantially similar binding properties. Such antibodies or fragments thereof comprise sequences 95%, 90%, 85%, or 80% identical to one or more of the CDR1. CDR2, or CDR3 heavy or light chain from FIGS. 9 and 10. The present invention also includes hybridoma lines and the monoclonal antibody molecules that they secrete, which are specific to TF and TFRC antigen expressed by normal or cancer cells. The present invention also includes chimeric antibodies, such as humanized, and antibody fragments and also includes other modified TF antibodies, TF antibody fragments, TFRC antibodies, and TFRC antibody fragments.

In addition to the specific amino acid sequences of the antigen binding sites of the heavy and light chains set forth in FIGS. 9 and 10, the present invention also encompasses TF antibodies, TF antibody fragments, TFRC antibodies, and TFRC antibody fragments that have preferential binding to TF or TFRC antigens but which have FWR and/or CDR antigen binding site amino acid sequences that are not identical to those set forth in FIGS. 9 and 10. Such TF antibodies, TF antibody fragments, TFRC antibodies, and TFRC antibody fragments are preferred if they are specific or preferentially selective for the TF or TFRC antigen, preferably at least 85% or more as specific, more preferably at least 90% or more as specific, and most preferably at least 95% or more as specific for the TF or TFRC antigen as the Alper-TF mAb or antibody fragment therefor. According to a preferred aspect, a variant of a TF antibody, TF antibody fragment, TFRC antibody, or TFRC antibody fragment of the present invention can be as specific for the TF or TFRC antigen as a non-variant antibody or antibody fragment of the present invention, or can be more specific.

TF antibodies and TF antibody fragments that are specific to TF but which have FWR and/or CDR antigen binding site amino acid sequences that are not identical to those set forth in FIGS. 9 and 10 can possess the same or different specificity determining regions (SDRs) as the FWRs and/or CDRs of FIGS. 9 and 10 (set forth in Tables 1 and 2). Similarly, TFRC antibodies and TFRC antibody fragments that are specific to TFRC but which have FWR and/or CDR antigen binding site amino acid sequences that are not identical to those set forth in FIGS. 9 and 10 can possess the same or different specificity determining regions (SDRs) as the FWRs and/or CDRs of FIGS. 9 and 10 (set forth in Tables 1 and 2).

Modifications to the amino acid sequences of the antigen binding sites CDR1, CDR2, and CDR3 set forth in FIG. 9 (heavy chain) and FIG. 10 (light chain) can occur in either or both of the FWR and CDR sequences. According to certain aspects of the invention, variations in antibodies or antibody fragments can occur where they have substantially homologous amino acid sequences, substantially similar binding properties, or both.

Humanized variants of the antibodies or antibody fragments of the invention can contain a reduced murine content, and potentially, reduced immunogenicity, when compared to murine antibodies, such as Alper-TF mAb, or antibody fragments thereof. Humanized variants include those that retain a binding affinity that is substantially similar to that of the original antibody or antibody fragment. An aspect of the invention provides CDR variants of humanized TF antibodies, TF antibody fragments, TFRC antibodies, or TFRC antibody fragments in which 1, 2, 3, 4, 5, or 6 (three heavy chain and three light chain) CDRs are humanized. A second aspect of the invention provides SDR variants of humanized TF antibodies, TF antibody fragments, TFRC antibodies, and TFRC antibody fragments in which only Specificity Determining Residues (SDRs) from the TF antibodies, TF antibody fragments, TFRC antibodies, and TFRC antibody fragments are present in the humanized antibodies. The SDRs are selected from Table 1 or Table 2.

TABLE 1
Specificity-Determining Residues in Alper-TF
mAb Heavy Chain (SEQ ID NO. 1).

| Position | Residue |
|---|---|
| 6 | C |
| 18 | G |
| 76 | N |
| 91 | T |
| 94 | F |
| 95 | C |

TABLE 2
Specificity-Determining Residues in Alper-TF
mAb Light Chain (SEQ ID NO. 5).

| Position | Residue |
|---|---|
| 3 | L |
| 18 | N |
| 48 | L |
| 50 | K |
| 51 | E |
| 59 | S |
| 74 | R |
| 94 | I |

CDR variants can be formed by replacing at least one CDR of a humanized TF antibody, TF antibody fragment, TFRC antibody, or TFRC antibody fragment with a corresponding CDR from a human antibody. CDR variants include those in which one, two, three, four, five, or six CDRs are replaced by a corresponding CDR from a human antibody and retain biological activity that is substantially similar to the binding affinity of the parental TF or TFRC mAb. CDR variants of the invention can have a binding affinity that is 25% more than the binding affinity of the parental TF or TFRC antibody or antibody fragment, more preferably more than 50%, and most preferably more than at least 75% or 90%.

CDR variants can have altered immunogenicity when compared to TF and TFRC antibodies and TF or TFRC antibody fragments can be formed by grafting all six (three heavy chain and three light chain) CDRs from the TF antibodies, TF antibody fragments, TFRC antibodies, and TFRC antibody fragments of the present invention onto the variable light (V_{L}) and variable heavy (V_{H}) frameworks of human TF antibodies, TF antibody fragments, TFRC antibodies, and TFRC antibody fragments. However, less than all six of the CDRs of the TF antibodies, TF antibody fragments, TFRC antibodies, and TFRC antibody fragments of the present invention can be present, while still permitting an antibody of the present invention to retain activity.

Residues that are directly involved in antigen contact, such as Specificity Determining Residues (SDRs), can be refined. SDR variants are formed by replacing at least one SDR of the TF antibody, TF antibody fragment, TFRC antibody, or TFRC antibody fragment with a residue at a corresponding position from a human antibody. It should be noted that not all CDRs must include SDRs.

In a preferred aspect, the variants of the present TF antibodies and TF antibody fragments include a combination of CDR and/or SDR substitutions to generate variants having reduced immunogenicity in humans and a binding affinity that is substantially similar to that of the parental antibody or antibody fragment to TF. In another aspect, the variants of the present TFRC antibodies and TFRC antibody fragments include a combination of CDR and/or SDR substitutions to generate variants having reduced immunogenicity in humans and a binding affinity that is substantially similar to that of the parental antibody or antibody fragment to TFRC.

In addition to variants specifically described herein, other “substantially homologous” modified immunoglobulins can be readily designed and manufactured using various recombinant DNA techniques. For example, the framework regions (FWRs) can be varied at the primary structure level. Moreover, a variety of different human framework regions can be used singly or in combination as a basis for the variant. In general, modifications of the genes can be readily accomplished by a variety of techniques, such as site-directed mutagenesis and random mutagenesis.

Alternatively, polypeptide fragments comprising only a portion of the primary antibody structure can be produced where the fragment substantially retains the immunoreactivity properties of the variant. Such polypeptide fragments include fragments produced by techniques known in the art, such as proteolytic cleavage of intact antibodies or fragments produced by inserting stop codons at the desired locations in the nucleotide sequence using site-directed mutagenesis. Single chain antibodies and fusion proteins which include at least an immunoreactivity fragment of the variant are also included within the scope of the invention.

TF antibodies, TF antibody fragments, TFRC antibodies, and TFRC antibody fragments can optionally be immobilized on a solid phase, detectably labeled, or conjugated to a cytotoxic radionuclide, a cytotoxic drug, or a cytotoxic protein and the like. Compositions comprising an Alper-TF mAb immobilized on a solid phase are encompassed.

The antibodies and their variants in accordance with the present invention can be directly or indirectly attached to effector moieties having therapeutic activity. Suitable effector moieties include but not limited to cytokines, cytotoxins, radionuclides, drugs, immunomodulators, therapeutic enzymes, and anti-proliferative agents. Methods for attaching antibodies to such effectors are known in the art. These conjugated antibodies can be incorporated into any composition, including pharmaceutical compositions for use in treating diseases characterized by the expression of TF and/or TFRC, including cancer, such as cancer of the breast, ovary, cervix, prostate, colon, stomach, kidney, liver, head, neck, lung, blood, pancreas, skin, testicle, thyroid and brain, most preferentially human breast, ovary, head, neck, brain, and prostate, in particular human prostate cancer. The pharmaceutical compositions are preferably administered to a mammal, more preferably a human patient in need of such treatment, in order to treat the disease. The antibodies useful in therapeutic applications are typically humanized and humanized Alper-TF mAbs are encompassed.

TF antibodies, TF antibody fragments, TFRC antibodies, and TFRC antibody fragments can either be labeled or unlabeled. Unlabeled antibodies can be used in combination with other labeled antibodies (secondary antibodies) that are reactive with the humanized antibody, such as antibodies specific for human immunoglobulin constant regions. Alternatively, the antibodies can be directly labeled. A wide variety of labels can be employed, such as radionuclides, fluors, enzymes, enzyme substrates, enzyme cofactors, enzyme inhibitors, ligands (particularly haptens), etc. Numerous types of immunoassays are available and known in the art.

In one embodiment, an isolated antibody that binds TF is contemplated. The isolated antibody comprises a heavy chain variable domain comprising three complementarity determining regions (CDRs) comprising the amino acid sequences of SEQ ID NO: 2, SEQ ID NO: 3, and SEQ ID NO: 4 and a light chain variable domain comprising three CDRs comprising the amino acid sequences of SEQ ID NO: 6, SEQ ID NO: 7, and SEQ ID NO: 8.

In another embodiment, an isolated antibody that binds TFRC is contemplated. The isolated antibody comprises a heavy chain variable domain comprising three complementarity determining regions (CDRs) comprising the amino acid sequences of SEQ ID NO: 2, SEQ ID NO: 3, and SEQ ID NO: 4 and a light chain variable domain comprising three CDRs comprising the amino acid sequences of SEQ ID NO: 6, SEQ ID NO: 7, and SEQ ID NO: 8.

In another aspect, an isolated antibody that binds TF is contemplated, wherein the antibody binds to the same epitope as an antibody comprising a heavy chain variable domain comprising the amino acids of SEQ ID NO: 1 and a light chain variable domain comprising the amino acids of SEQ ID NO: 5.

In yet another aspect, an isolated antibody that binds TFRC is contemplated, wherein the antibody binds to the same epitope as an antibody comprising a heavy chain variable domain comprising the amino acids of SEQ ID NO: 1 and a light chain variable domain comprising the amino acids of SEQ ID NO: 5.

Also encompassed is an isolated antibody that comprises a heavy chain variable domain comprising the amino acids of SEQ ID NO: 1 and a light chain variable domain comprising the amino acids of SEQ ID NO: 5.

The isolated antibody of the invention recognizes a soluble protein having a molecular weight of about 77 kDa as measured by gradient polyacrylamide gel electrophoresis.

The isolated antibody is capable of binding to a precursor form of TF with a specific affinity of between 10⁻⁸ M and 10⁻¹¹ M.

The isolated antibody is also capable of binding to a mature form of TF with a specific affinity of between 10⁻⁸ M and 10⁻¹¹ M.

In another aspect, the isolated antibody of the invention recognizes a soluble protein having a molecular weight of about 85 kDa as measured by gradient polyacrylamide gel electrophoresis.

The isolated antibody is capable of binding to a precursor form of TFRC with a specific affinity of between 10⁻⁸ M and 10⁻¹¹ M.

The isolated antibody is also capable of binding to a mature form of TFRC with a specific affinity of between 10⁻⁸ M and 10⁻¹¹ M.

Encompassed is an isolated antibody that recognizes at least one epitope selected from the group consisting of the amino acids of SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 28, SEQ ID NO: 29, SEQ ID NO: 30, and SEQ ID NO: 31, or fragments of these amino acids.

An isolated antibody described herein that is immobilized on a solid phase is contemplated.

The isolated antibody described herein may be conjugated to an agent selected from the group consisting of: a detectable label, a cytotoxic radionuclide, a cytotoxic drug, and a cytotoxic protein.

An isolated DNA molecule which encodes the antibody described herein, as well as isolated vectors comprising DNA that encodes the heavy and/or light chain described herein is encompassed.

A kit comprising: an isolated antibody comprising a heavy chain variable domain comprising three complementarity determining regions (CDRs) comprising the amino acid sequences of SEQ ID NO: 2, SEQ ID NO: 3, and SEQ ID NO: 4 and a light chain variable domain comprising three CDRs comprising the amino acid sequences of SEQ ID NO: 6, SEQ ID NO: 7, and SEQ ID NO: 8 and a secondary antibody that binds to the antibody, wherein the secondary antibody is conjugated to a detectable label is encompassed.

A composition comprising a tissue specimen and an antibody-antigen complex between the antibody described herein and TF within the tissue specimen is encompassed.

A composition comprising a tissue specimen and an antibody-antigen complex between the antibody described herein and TFRC within the tissue specimen is encompassed.

A composition comprising a tissue specimen and an antibody-antigen complex between the antibody described herein and an antigen recognized by an Alper-TF mAb within the tissue specimen is encompassed.

A pharmaceutical composition comprising the antibody described herein in combination with a pharmaceutically acceptable carrier is contemplated.

In some embodiments, the pharmaceutical composition is administered to a subject in need thereof intravenously, intramuscularly, intraarterially, intrathecally, intracapsularly, intraorbitally, intracardiacally, intradermally, intraperitoneally, transtracheally, subcutaneously, subcuticularly, intraarticularly, subcapsularly, subarachnoidally, intraspinally, epidurally, and intrasternally.

In another aspect, the disclosure features a method of modulating interaction between TF and TFRC. For example, an anti-TFRC antibody can be used to reduce or inhibit binding, between TF and TFRC. The method can be used on cells in vitro or ex vivo. For example, TFRC receptor-expressing cells can be cultured in vitro in culture medium and the contacting step can be affected by adding an anti-TFRC antibody to the culture medium. Alternatively, the method can be performed on cells present in a subject, e.g., as part of an in vivo (e.g., therapeutic or prophylactic) protocol. For example, the anti-TFRC antibody can be delivered locally or systemically. In one embodiment, an anti-TFRC antibody described herein is used for the preparation of a medicament for of modulating interaction between TF and TFRC.

The method can include contacting TF with TFRC, under conditions that allow an interaction between TF and TFRC, to occur to thereby form TF/TFRC mixture. Generally, the anti-TFRC antibody is provided in an effective amount so that contacting the TF/TFRC with the anti-TFRC antibody modulates (e.g., interferes with, inhibits, blocks or otherwise reduces) the interaction between TF and TFRC or at least one function of TFRC, e.g., TFRC mediated signaling.

Nucleic Acid Molecules and Host Cells

Any of the antibodies or antibody fragments of the present invention can be encoded by nucleic acids. The present invention includes such molecules, fragments of such molecules, and such molecules included in vectors and the like. Nucleic acid molecules also include the complement of such nucleic acid molecules. Both DNA and RNA molecules are examples of nucleic acid molecules.

In another aspect, the present invention provides an isolated DNA sequence which encodes the heavy chain of an antibody molecule, where the antibody molecule has preferential binding for TF or TFRC antigens, including at least TF or TFRC, and where the variable domain of the heavy chain comprises a CDR having the antigen binding site amino acid sequences of at least one, two, or all three CDR1, CDR2, and CDR3 set forth in FIG. 9.

In yet another aspect, the present invention provides an isolated DNA sequence which encodes the light chain of an antibody molecule, where the antibody molecule has preferential binding for TF or TFRC antigens, including at least TF or TFRC, and further where the variable domain of the light chain comprises a CDR having the antigen binding site amino acid sequences of at least one, two or all three CDR1, CDR2, and CDR3 set forth in FIG. 10.

In another aspect, the present invention includes a nucleic acid molecule in a host cell. Such nucleic acid molecule can be integrated into the genome of the host cell or can be present on a vector such as a plasmid or viral vector. A nucleic acid molecule of the present invention may be transiently present in such a host cell. In some embodiments, a host cell is selected from the group consisting of E. coli; Bacilli, (e.g., Bacillus subtilis); enterobacteriacae (e.g., Salmonella, Serratia and Pseudomonas); yeast (e.g., Saccharomyces; Pichia pastoris); Sf9 insect cells; Sp2/0 cells; VERO cells; HeLa cells; Chinese hamster ovary (CHO) cells; W138 cells; BHK cells; COS-7 cells; and MDCK cells. In other embodiments, a host cell is selected from a breast cancer cell line such as SKBR3, MCF-7, MDA-MB-231, MDA-MB-435, and ZR75B. In another aspect, a host cell is selected from a prostate cancer cell line such as PC3, DU145 and LNCap.

Methods of Making TF Antibodies or Antibody Fragments

TF antibodies, TF antibody fragments, TFRC antibodies, and TFRC antibody fragments of the present invention can be developed, for example, using the human prostate cancer cell line OPCT1, derived from prostate tumor epithelium resected from a patient who received no chemotherapy, radiotherapy, or hormone treatment (T1cN0M0; Gleason 3+3; available from Asterand Inc.).

The present invention includes processes for producing monoclonal chimeric antibodies, including humanized, using recombinant DNA technology. See, for example, Antibodies, A Laboratory Manual (Harlow & Lane Eds., Cold Spring Harbor Press, 1988), which is herein incorporated by reference in its entirety.

TF antibodies, TF antibody fragments, TFRC antibodies, or TFRC antibody fragments of the present invention can be produced by any known method including, without limitation, generating murine hybridomas which produce antibodies or antibody fragments specific for TF or TFRC. Hybridomas can be formed, for example, by the fusion of a mouse fusion partner cell and spleen cells from mice immunized against native TF or native TFRC prepared without fixation. Mice can be also immunized with crude or semi-purified preparations containing an antigen of interest, such as a native TF or native TFRC isolated without fixation. To immunize the mice, a variety of different conventional protocols can be followed. For example, mice can receive primary and boosting immunizations of antigenic preparations.

Cell fusions can be accomplished by any procedures known to those skilled in the field of immunology. Fusion partner cell lines and methods for fusing and selecting hybridomas and screening for antibodies or antibody fragments are known.

Antibodies or antibody fragments of the present invention can be produced in large quantities, for example, by injecting hybridoma cells secreting the antibody into the peritoneal cavity of mice and, after appropriate time, harvesting the ascites fluid which contains a high titer of the antibody or antibody fragment, and isolating the antibody or antibody fragment therefrom. Alternatively, the TF antibodies, TF antibody fragments, TFRC antibodies, or TFRC antibody fragments can be produced by culturing hybridoma cells in vitro and isolating the secreted antibody or antibody fragment from the cell culture medium.

TF antibodies, TF antibody fragments, TFRC antibodies, or TFRC antibody fragments of the present invention can also be produced by expressing the appropriate DNA sequence in a host after the sequence has been operably linked to an expression control sequence. Such expression vectors are often replicable in a host organism either as episomes or as an integral part of the host chromosomal DNA. Expression vectors often contain expression control sequences compatible with the host cell, such as an origin of replication. In addition, an expression vector can include a promoter to control expression of the gene, optionally, with operator sequences, and have ribosome binding site sequences and the like for initiating and completing transcription and translation. Suitable promoters include, without limitation, the polyhedrin promoter, lactose promoter system, a tryptophan promoter system, a beta-lactamase promoter system, or a promoter system from phage lambda. Expression vectors can also contain selection markers. DNA sequences encoding the light chain and heavy chain of a TF antibody or antibody fragments can be inserted into separate expression vectors, or into the same expression vector.

Suitable hosts include, without limitation, prokaryotic strains such as E. coli; Bacilli, including Bacillus subtilis; enterobacteriacae, including Salmonella, Serratia and Pseudomonas. Suitable hosts also include eukaryotic hosts such as yeast, including Saccharomyces; Pichia pastoris; Sf9 insect cells; Sp2/0, VERO and HeLa cells, Chinese hamster ovary (CHO) cell lines; W138, BHK, COS-7 and MDCK cell lines. Other suitable hosts can also be used in accordance with known expression techniques.

The vectors containing the DNA segments of interest can be transferred into the host cell by any method, which varies depending on the type of cellular host. For example, calcium chloride transfection, calcium phosphate treatment, electroporation or cationic liposome mediated transfection (such as DOTAP). Successfully transformed cells, can be identified by a variety of techniques for detecting the binding of a receptor to a ligand.

Expressed gene products can be purified according to any method, including, without limitation, ammonium sulfate precipitation, affinity columns, column chromatography, and gel electrophoresis. Substantially pure immunoglobulins of at least 80% homogeneity are preferred, with about 90% to about 95% homogeneity being more preferred, and 98% to 99% or more homogeneity is most preferred, and is generally considered acceptable for pharmaceutical uses.

Isolated or purified DNA sequences can be incorporated into a cloning or expression vector, which can in turn be used to transform a host cell. The transformed host cells can be used in a process for the production of an antibody molecule having specificity for TF or TFRC antigens, including culturing the host cells and Isolating the antibody molecules they produce.

Diagnostic Methods, Assays, and Kits

The antibodies of the present invention can be used in any type of immunoassay to detect a disease or disorder characterized by aberrant expression of TF or TFRC. Non-limiting examples include cancer, prostate cancer, iron deficiency anemia, and neurological disorders.

In one embodiment, an in vitro method for detecting TF in a biological sample, comprising: (a) contacting a biological sample with an Alper-TF mAb; and (b) qualitatively or quantitatively determining the formation of an immune complex between the antibody and TF, is encompassed. The biological sample may be from a human subject in need of diagnosis of prostate cancer. The biological sample may be from a human subject diagnosed with prostate cancer. The formation of an immune complex between the antibody and TF indicates the presence of cancer. The biological sample may be selected from tissue, cells, blood, serum, plasma, urine, and exosomes purified from blood, serum, plasma, urine, and exosomes purified from urine.

In another embodiment, an in vitro method for detecting TFRC in a biological sample, comprising: (a) contacting a biological sample with an Alper-TF mAb; and (b) qualitatively or quantitatively determining the formation of an immune complex between the antibody and TFRC, is encompassed. The biological sample may be from a human subject in need of diagnosis of prostate cancer. The biological sample may be from a human subject diagnosed with prostate cancer. The formation of an immune complex between the antibody and TF indicates the presence of cancer. The biological sample may be selected from tissue, cells, blood, serum, plasma, urine, and exosomes purified from urine.

In another embodiment, an in vitro method for diagnosing early and late-stage prostate cancer in a human subject comprising: (a) isolating a tissue or cell sample from a subject; (b) contacting the tissue or cell sample with an Alper-TF mAb; (c) labeling the sample with an agent that detects the antibody; (d) visualizing the location of the labeled antibody within the tissue or cell; and (e) diagnosing early stage prostate cancer if the labeled antibody is located within an endosome, and diagnosing late stage prostate cancer if the labeled antibody not within an endosome is contemplated.

In another aspect, a method for diagnosing cancer in humans comprising: (a) removing a specimen from a patient suspected of having a cancer; (b) contacting the specimen with an effective binding amount of an Alper-TF mAb, thereby forming antigen-antibody complexes in said specimen; (c) detecting the antigen-antibody complex; and (e) diagnosing cancer if at least one antigen-antibody complex is detected is encompassed.

The cancer may be selected from the group consisting of human breast, prostate, ovary, head, neck, and brain.

In a further aspect, the present invention includes an immunoassay for preferentially detecting a TF antigen preferentially bound by an Alper-TF mAb, where the assay comprises using a TF antibody or TF antibody fragment of the present invention. In yet a further aspect, the present invention includes an immunoassay for preferentially detecting a TFRC antigen preferentially bound by an Alper-TF mAb, where the assay comprises using a TFRC antibody or TFRC antibody fragment of the present invention.

The present invention also includes an assay for preferentially detecting one or more TF antigens, including a TF antigen, which binds to a monoclonal antibody having one or more of the heavy chain CDR antigen binding site amino acid sequences set forth in FIG. 9, such as SEQ ID NOs: 2-5, and one or more of the light chain CDR antigen binding site amino acid sequences set forth in FIG. 10, such as SEQ ID NOs: 6-8. The detection can be in vitro or in vivo.

The present invention also includes an assay for preferentially detecting one or more TFRC antigens, including a TFRC antigen, which binds to a monoclonal antibody having one or more of the heavy chain CDR antigen binding site amino acid sequences set forth in FIG. 9, such as SEQ ID NOs: 2-5, and one or more of the light chain CDR antigen binding site amino acid sequences set forth in FIG. 10, such as SEQ ID NOs: 6-8. The detection can be in vitro or in vivo.

Such assays can be used in any suitable manner, including, without limitation, by comprising: (a) contacting the sample with an effective binding amount of one of the TF antibodies or TF antibody fragments of the invention; and (b) detecting the TF antigen by detecting the preferential binding of the antibody to a TF antigen. Assays of the present invention can be used to detect cancer in tissues, cells, blood, serum, plasma, or urine. The immunoassay can detect TF, including, TF that has been post-transcriptionally processed, and a soluble/secreted precursor TF.

Such assays can also be used in any suitable manner, including, without limitation, by comprising: (a) contacting the sample with an effective binding amount of one of the TFRC antibodies or TFRC antibody fragments of the invention; and (b) detecting the TFRC antigen by detecting the preferential binding of the antibody to a TFRC antigen. Assays of the present invention can be used to detect cancer in tissues, cells, blood, serum, plasma, or urine. The immunoassay can detect TFRC, including TFRC that has been post-transcriptionally processed, and a soluble/secreted precursor TFRC.

In a further aspect, the present invention provides a kit for the immunocytochemical detection, including but not limited to immunohistochemical or immunofluorescent detection, of carcinoma comprising: (a) a TF antibody or TF antibody fragment of the present invention, such as Alper-TF mAb; and (b) a secondary antibody conjugated to a detectable label. In some embodiments, the detection can be in vitro and is for prostate cancer detection.

In yet a further aspect, the present invention provides a kit for the immunocytochemical detection, including but not limited to immunohistochemical or immunofluorescent detection, of carcinoma comprising: (a) a TFRC antibody or TFRC antibody fragment of the present invention, such as Alper-TF mAb; and (b) a secondary antibody conjugated to a detectable label. In some embodiments, the detection can be in vitro and is for prostate cancer detection.

The present invention includes a kit with a TF antibody or TF antibody fragment of the present invention, such as Alper-TF mAb, that detects a TF antigen preferentially bound by Alper-TF mAb in the early endosome, most preferably in prostate cancer cells of early stage prostate cancer subjects. The TF antigen preferentially bound by Alper-TF mAb is localized to the late endosomes in prostate cells, most preferably in subjects with later stages of prostate cancer. In one aspect, levels of soluble TF antigen in late endosomes of prostate cancer cells are significantly associated with decreased chance of survival relative to the chance of survival of patients with soluble TF antigen in early endosomes of prostate cancer cells, observed in patients with early-stage prostate cancer. In yet another aspect, the TF antibody or TF antibody fragment included in the kit preferentially binds TF antigen in exosomes, preferably exosomes located in the extracellular space, blood, plasma, serum, or urine.

The present invention also includes a kit with a TFRC antibody or TFRC antibody fragment of the present invention, such as Alper-TF mAb, that detects a TFRC antigen preferentially bound by Alper-TF mAb in the early endosome, most preferably in prostate cancer cells of early stage prostate cancer subjects. The TFRC antigen preferentially bound by Alper-TF mAb is localized to the late endosomes in prostate cells, most preferably in subjects with later stages of prostate cancer. In one aspect, levels of TFRC antigen in late endosomes of prostate cancer cells are significantly associated with decreased chance of survival relative to the chance of survival of patients with TFRC antigen in early endosomes of prostate cancer cells, observed in patients with early-stage prostate cancer. In yet another aspect, the TFRC antibody or TFRC antibody fragment included in the kit preferentially binds TFRC antigen in exosomes, preferably exosomes located in the extracellular space, blood, plasma, serum, or urine.

In a further aspect, the present invention provides a kit comprising a TF antibody, TFRC antibody, TF antibody fragment, and/or TFRC antibody fragment; and a secondary antibody conjugated to a detectable label. In some embodiments, the detection can be in vitro and is for prostate cancer detection.

In a further aspect, the present invention provides a kit for the immunocytochemical detection, including but not limited to immunohistochemical or immunofluorescent detection, of carcinoma comprising: (a) a monoclonal antibody having one or more of the heavy chain CDR antigen binding site amino acid sequences set forth in FIG. 9, such as SEQ ID NOs: 2-5, and one or more of the light chain CDR antigen binding site amino acid sequences set forth in FIG. 10, such as SEQ ID NOs: 6-8; and (b) a secondary antibody conjugated to a detectable label.

All of the kits described herein may include reagents for assaying a sample for a TF or TFRC antigen, such as, for example, buffers, instructions, TF or TFRC antigen specific affinity reagents, such as an antibody, or fragment or mimetic thereof, and/or immunoassay devices comprising the same members of a signal producing system, such as antibodies, enzyme substrates, and the like; various buffers for use in carrying out the subject detection assays; a reference for determining the amount of one or more TF or TFRC antigens in a sample; and the like. Other examples of kits or kit formats are found in Alper, US Publication No. 2008/0293162, herein incorporated by reference in its entirety.

In a further aspect, the present invention provides a method for diagnosing cancer, such as prostate cancer, in humans comprising: (a) removing a specimen from a patient suspected of having a cancer; (b) contacting the specimen with a TF antibody, TFRC antibody, TF antibody fragment, or TFRC antibody fragment of the present invention; (c) labeling the specimen; and (d) detecting the presence of the antigen-antibody complex by the label. Detection of at least one antigen-antibody complex indicates a diagnosis of cancer. In an aspect, the specimen can be one or more of a tissue sample, cell sample, blood, serum, plasma, and urine. In an aspect, a cancer subject may have a greater amount of TF antigen in serum than in plasma of the same subject. In another aspect, a cancer subject may have a greater amount of TFRC antigen in serum than in plasma of the same subject. The difference in amount may be at least one order of magnitude to three orders of magnitude. The cancer may be selected from the group consisting of cancers of breast, ovary, cervix, prostate, colon, stomach, kidney, liver, head, neck, lung, blood, pancreas, skin, testis, thyroid and brain. In some embodiments, a prostate cancer subject may have a greater amount of TF antigen in his urine than in the urine of a healthy subject. In yet another aspect, a prostate cancer subject may have a greater amount of TFRC antigen in his urine than in the urine of a healthy subject.

In a still further aspect, the present invention provides a method for diagnosing prostate cancer in humans comprising: (a) removing a specimen from a patient suspected of having a prostate cancer; (b) contacting the specimen with a monoclonal antibody having one or more of the heavy chain CDR antigen binding site amino acid sequences set forth in FIG. 9, such as SEQ ID NOs: 2-5, and one or more of the light chain CDR antigen binding site amino acid sequences set forth in FIG. 10, such as SEQ ID NOs: 6-8; (c) labeling the specimen: and (d) detecting the presence of an increase in antigen-antibody complex by the label in the prostate cancer specimen compared to a specimen from a normal subject without prostate cancer. In an aspect, the specimen can be at least one of a tissue sample, blood, serum, plasma, and urine.

The cancers being diagnosed include, without limitation, those that are selected from the group consisting of breast, ovary, cervix, prostate, colon, stomach, kidney, liver, head, neck, lung, pancreas, skin, testicle, thyroid and brain cancer, in particular human prostate cancer.

In an aspect. TF levels are higher in prostate patients relative to age-matched healthy controls. The increase in prostate cancer patients compared to age-matched healthy controls may be at least about 200%, about 300%, about 400%, about 500% or about 600% in their plasma levels of a TF form preferentially bound by Alper-TF mAb. In another aspect, TF levels are higher in late-stage prostate cancer patients relative to age-matched healthy controls or an early-stage prostate cancer subject. In a third aspect, TF levels are higher in late-stage prostate cancer patients relative to age-matched healthy controls. In one aspect, the levels of TF are higher in early-stage prostate cancer patients relative to age-matched healthy controls. Similarly, the levels of TF are higher in last stage prostate cancer relative to age-matched healthy controls. An increase in TF levels can mean that they are statistically significant relative to age-matched healthy controls. Levels of TF similar to that of healthy control can mean that the levels are not statistically significant. In an aspect, the statistically significant differences in levels of TF have a p-value of p<0.05 as measured by an appropriate statistical test, such as the student's T-test or the Mann-Whitney test. In another aspect, the statistically significant differences in levels of TF have a p-value of p<0.01 as measured by an appropriate statistical test, such as the student's T-test or the Mann-Whitney test. In a further aspect, the statistically significant differences in levels of TF have a p-value of p<0.005 as measured by an appropriate statistical test, such as the student's T-test or the Mann-Whitney test. In a further aspect, the statistically significant differences in levels of TF have a p-value of p<0.001 as measured by an appropriate statistical test, such as the student's T-test or the Mann-Whitney test.

In one aspect, TFRC levels are higher in prostate patients relative to age-matched healthy controls. The increase in prostate cancer patients compared to age-matched healthy controls may be at least about 200%, about 300%, about 400%, about 500% or about 600% in their plasma levels of a TFRC form preferentially bound by Alper-TF mAb. In another aspect, TFRC levels are higher in late-stage prostate cancer patients relative to age-matched healthy controls or an early-stage prostate cancer subject. In a third aspect, TFRC levels are higher in late-stage prostate cancer patients relative to age-matched healthy controls. In one aspect, the level of TFRC is higher in early-stage prostate cancer patients relative to age-matched healthy controls. Similarly, the levels of TFRC are higher in last stage prostate cancer relative to age-matched healthy controls. An increase in TFRC levels can mean that they are statistically significant relative to age-matched healthy controls. Levels similar to healthy control levels can mean that the levels are not statistically significant. In an aspect, the statistically significant differences in levels of TFRC have a p-value of p<0.05 as measured by an appropriate statistical test, such as the student's T-test or the Mann-Whitney test. In another aspect, the statistically significant differences in levels of TFRC have a p-value of p<0.01 as measured by an appropriate statistical test, such as the student's T-test or the Mann-Whitney test. In a further aspect, the statistically significant differences in levels of TFRC have a p-value of p<0.005 as measured by an appropriate statistical test, such as the student's T-test or the Mann-Whitney test. In a further aspect, the statistically significant differences in levels of TFRC have a p-value of p<0.001 as measured by an appropriate statistical test, such as the student's T-test or the Mann-Whitney test.

In a further aspect, the present invention provides a method for diagnosing prostate cancer in a subject in need thereof comprising: (a) contacting a specimen from said subject with a TF antibody or TF antibody fragment of the present invention; (b) labeling the specimen; and (c) detecting an increase of TF in a patient with prostate cancer, where such prostate cancer can be in early-stage, mid-stage, or late-stage, most preferably, early- or mid-stage prostate cancer. In an aspect, the specimen can be at least one of a tissue, blood, serum, plasma, and urine. The detection can be in vitro or in vivo.

In yet a further aspect, the present invention provides a method for diagnosing prostate cancer in a subject in need thereof comprising: (a) contacting a specimen from said subject with a TFRC antibody or TFRC antibody fragment of the present invention; (b) labeling the specimen; and (c) detecting an increase of TFRC in a patient with prostate cancer, where such prostate cancer can be in early-stage, mid-stage, or late-stage, most preferably, early- or mid-stage prostate cancer. In an aspect, the specimen can be at least one of a tissue, blood, serum, plasma, and urine. The detection can be in vitro or in vivo.

In a still further aspect, the present invention provides a method for diagnosing prostate cancer in humans comprising: (a) removing a specimen from a patient suspected of having a cancer; (b) contacting the specimen with a monoclonal antibody having one or more of the heavy chain CDR antigen binding site amino acid sequences set forth in FIG. 9, such as SEQ ID NOs: 2-5, and one or more of the light chain CDR antigen binding site amino acid sequences set forth in FIG. 10, such as SEQ ID NOs: 6-8; (c) labeling the specimen; and (d) detecting the presence of the antigen-antibody complex by the label.

The cancer being assayed, diagnosed, evaluated, monitored and/or predicted can be any of early-, mid- or late-stage prostate cancer or a combination thereof.

Without limitation, the biological sample for all methods and uses described herein include tissue, cell, blood, serum, plasma, urine and exosomes in the urine.

In an additional aspect, the present invention includes a method for developing drugs useful in treating, diagnosing, or both treating and diagnosing diseases characterized by the expression of gene products of TF and homologues thereof, including identifying gene products expressed by TF and homologues thereof, and utilizing the gene products as biomarkers in the development and identification of drugs selected from the group comprising TF antibodies and TF antibody fragments, inhibiting peptides, siRNA, antisense oligonucleotides, vaccines, and chemical compounds, which specifically target the gene products.

In an additional aspect, the present invention includes a method for developing drugs useful in treating, diagnosing, or both treating and diagnosing diseases characterized by the expression of gene products of TFRC and homologues thereof, including identifying gene products expressed by TFRC and homologues thereof, and utilizing the gene products as biomarkers in the development and identification of drugs selected from the group comprising TFRC antibodies and TFRC antibody fragments, inhibiting peptides, siRNA, antisense oligonucleotides, vaccines, and chemical compounds, which specifically target the gene products.

A TF antibody or TF antibody fragment of the present invention can be used in diagnosis of diseases characterized by the expression of TF, such as cancer. For example, in vivo diagnosis and imaging of a solid tumor of the breast, ovary, cervix, prostate, colon, stomach, kidney, liver, head, neck, lung, blood, pancreas, skin, testicle, thyroid or brain, and combinations thereof, most preferentially human prostate cancer cells that express TF can be performed in accordance with the methods of the invention. A TF antibody or TF antibody fragment of the present invention can also be used for diagnosis in vitro, for example, by using a TF antibody or TF antibody fragment to detect the presence of the cancer marker TF in a fluid sample, such as a tissue sample, plasma, serum, or urine.

A TFRC antibody or TFRC antibody fragment of the present invention can also be used in diagnosis of diseases characterized by the expression of TFRC, such as cancer. For example, in vivo diagnosis and imaging of a solid tumor of the breast, ovary, cervix, prostate, colon, stomach, kidney, liver, head, neck, lung, blood, pancreas, skin, testicle, thyroid or brain and combinations thereof, most preferentially human prostate cancer cells that express TFRC can be performed in accordance with the methods of the invention. A TFRC antibody or TFRC antibody fragment of the present invention can also be used for diagnosis in vitro, for example, by using a TFRC antibody or TFRC antibody fragment to detect the presence of the cancer marker TFRC in a fluid sample, such as a tissue sample, plasma, serum, or urine.

TF antibodies, TFRC antibodies, TF antibody fragments, and TFRC antibody fragments can be used in immunoassays to screen body fluids, such as serum, sputum, effusions, urine, cerebrospinal fluid, and the like, for the presence of TF or TFRC. TF antibodies, TFRC antibodies, TF antibody fragments, and TFRC antibody fragments can be used for scanning or radioimaging, when labeled with an appropriate radiolabel, to detect primary or metastatic foci of tumor cells. Furthermore, the antibodies are useful in lymphoscintigraphy to detect lymph node involvement in the disease.

A TF antibody, TFRC antibody, TF antibody fragment, and TFRC antibody fragment, which can include any or all of the antibodies or antibody fragments specific for TF or TFRC-related gene products, and/or chimeric antibodies or antibody fragments, such as humanized or other variants thereof, can be used therapeutically, or in developing and performing assays, in vivo or in vitro diagnostic procedures, and imaging. The antibodies can be used alone or in combination with a pharmaceutically-acceptable or diagnostic carrier formulation. TF antibodies, TFRC antibodies, TF antibody fragments, or TFRC antibody fragments can be incorporated into a pharmaceutically or diagnostically acceptable, non-toxic, sterile carrier as a suspension or solution. They can be used as separately administered compositions or given in conjunction with chemotherapeutic or immunosuppressive agents.

The present invention includes therapeutic, diagnostic, or therapeutic and diagnostic compositions comprising a TF antibody. TFRC antibody, TF antibody fragment, and TFRC antibody fragment of the present invention in combination or not with a pharmaceutically acceptable excipient, diluent, or carrier. The present invention also includes a process for preparation of a therapeutic or diagnostic composition comprising admixing an antibody molecule of the present invention together with a pharmaceutically acceptable excipient, diluent, or carrier. An antibody molecule can be the sole active ingredient in the therapeutic or diagnostic composition, or can be accompanied by other active ingredients including other antibody ingredients such as anti-T cell, anti-IFNγ, or anti-LPS antibodies, or non-antibody ingredients such as xanthines. Compositions can be incorporated into kits for diagnosing or treating diseases characterized by the expression of TF or TFRC, including, without limitation, solid tumors, and particularly solid tumors of the breast, ovary, cervix, prostate, colon, stomach, kidney, liver, head, neck, lung, pancreas, skin, testicle, thyroid and brain, most preferentially human prostate tumors.

Antibodies or antibody fragments of the present invention are useful for immunoassays which detect or quantitate a TF or TFRC form bound preferentially by Alper-TF mAb or cells secreting such a TF or TFRC in a sample. Such an immunoassay typically comprises incubating a biological sample from a subject with a need therefor, such as a man over 40-years old, in the presence of a detectably labeled antibody of the present invention capable of identifying the tumor antigen, and detecting the labeled antibody which is bound in a sample.

In an aspect of the present invention, the status of prostate cancer in a subject can be based on the relative amount, localization or both of one or more forms of TF or TFRC, including a TF or TFRC bound preferentially by Alper-TF mAb, in a blood, serum, plasma, or urine sample from a subject in need thereof as compared to that of a normal healthy age-matched subject. In one aspect, that status of cancer is whether the cancer cells are metastatic tumor cells, non-metastatic tumor cells, in particular from prostate cancer cells.

Examples of confirmatory analysis, assays, tests, such as histological examination of samples, and so forth that can be used to confirm or in combination with those disclosed herein include, without limitation, those set forth in Alper, US Publication No. 2008/0293162.

In an aspect of the present invention the level, localization, or both of one or more forms of TF or TFRC is diagnostic or prognostic of a disease or outcome probability.

TF antibodies, TFRC antibodies, TF antibody fragments, and TFRC antibody fragments of the present invention are also useful for immunopathological analysis, such as the differential diagnosis of tumor type and the subclassification of the tumor based on its expression or localization of at least one form of TF or TFRC, including, without limitation, assessment of metastatic potential, predicted responses to therapy, and overall prognosis.

TF antibodies, TFRC antibodies, TF antibody fragments, and TFRC antibody fragments permit the definition of subpopulations of tumor cells among the heterogeneous cells present in a growing tumor and can be used, for example, in the typing and cross-matching of the tumor cell “lines”, including, without limitation, by means of flow cytometry, both at the time of surgery and prior to therapy. An analysis of the tumor cell populations or subpopulations with antibodies or antibody fragments of this invention, and a battery of additional antibodies or antibody fragments, can be used to define (a) which antigen preparation would be the most appropriate for specific active immunotherapy; (b) which antibody or antibody fragment or chimeric antibody would be efficacious for the particular cancer; and (c) which antibody or combination of antibodies or antibody fragments should be used for imaging the patient at a later date in search for recurrent or metastatic tumors.

A biological sample can be treated with nitrocellulose, or other solid support or carrier which is capable of immobilizing cells, cell particles, soluble proteins, or glycoproteins. The support can then be washed with suitable buffers followed by treatment with the detectably labeled antibody of the present invention. The solid phase support can then be washed with the buffer a second time to remove unbound antibody. The amount of bound label on the solid support can then be detected by conventional means.

One of the ways in which the antibody of the present invention can be detectably labeled is by linking the same to an enzyme and use in an enzyme immunoassay (EIA) or enzyme-linked immunosorbent assay (ELISA). This enzyme, when subsequently exposed to its substrate, will react with the substrate generating a chemical moiety which can be detected, for example, by spectrophotometric, fluorometric, or visual means. In an alternate embodiment, the enzyme is used to label a binding partner for the antibody of the invention. Such a binding partner can be an antibody against the constant or variable region of the antibody of the invention, such as a heterologous anti-mouse immunoglobulin antibody. Alternatively, the binding partner can be a non-antibody protein capable of binding to the antibody of the present invention.

By radioactively labeling the antibodies of the present invention, it is possible to detect TF or TFRC through the use of a radioimmunoassay (RIA). The radioactive isotope can be detected by such means as the use of a gamma counter or a scintillation counter or by autoradiography. Isotopes which are particularly useful for the purpose of the present invention are known in the art.

It is also possible to label the antibodies of the present invention with a fluorescent compound. When the fluorescently labeled antibody is exposed to light of the proper wavelength, its presence can then be detected due to fluorescence. The antibodies of the present invention also can be detectably labeled by coupling to a chemiluminescent compound. The presence of the chemiluminescently labeled antibody is then determined by detecting the presence of luminescence that arises during the course of a chemical reaction. A bioluminescent compound can also be used to label the antibodies of the present invention. Bioluminescence is a type of chemiluminescence found in biological systems, in which a catalytic protein increases the efficiency of the chemiluminescent reaction. The presence of a bioluminescent protein is determined by detecting the presence of luminescence. Important bioluminescent compounds for purposes of labeling are luciferin, luciferase, and aequorin.

Detection of the antibody, fragment, or derivative can be accomplished by a scintillation counter, for example, if the detectable label is a radioactive gamma emitter, or by a fluorometer, for example, if the label is a fluorescent material. In the case of an enzyme label, the detection can be accomplished by colorimetric methods which employ a substrate for the enzyme. Detection can also be accomplished by visual comparison of the extent of enzymatic reaction of a substrate in comparison with similarly prepared standards.

In situ detection can be accomplished by removing a specimen from a patient, and providing the labeled antibody or the unlabeled antibody plus a labeled binding partner to such a specimen. Through the use of such a procedure, it is possible to determine not only the presence of the antigen but also its distribution in the examined tissue. Using the present invention, those of ordinary skill will readily perceive that any of a wide variety of histological methods (such as staining procedures) can be modified in order to achieve such in situ detection. Such methods include, for example, an immunocytochemical assay, including but not limited to an immunohistochemical or immunofluorescence assay. In an aspect, an avidin-biotin immunoperoxidase staining system can be used, and a kit utilizing this system is also contemplated, although the methods of the present invention can utilize any suitable staining procedures known in the art.

Kits according to the present invention can include frozen or lyophilized antibodies to be reconstituted by thawing or by suspension in a liquid vehicle. The kits can also include a carrier or buffer. Preferably, the kit also comprises instructions for reconstituting and using the antibody. The kit employing antibodies, including chimeric and humanized antibodies of the present invention, can be used for an immunocytochemical evaluation, including but not limited to an immunohistochemical or immunofluorescence assay, of cancers, including cancer of the breast, ovary, cervix, prostate, colon, stomach, kidney, liver, head, neck, lung, blood, pancreas, skin, testicle, thyroid and brain, most preferentially human breast, ovary, head, neck, brain, and prostate, in particular human prostate cancer.

The kits including the reagents necessary for an immunocytochemical analysis, including but not limited to an immunohistochemical or immunofluorescence analysis, can be provided as follows: a) a TF antibody, TFRC antibody, TF antibody fragment, or TFRC antibody fragment, or chimeric or humanized variants thereof; b) blocking reagent (in the form of, for example, goat serum) and secondary antibody (such as, for example, goat anti-mouse antibody); c) detectable marker (such as, for example, immunoperoxidase or alkaline phosphatase); and d) developing reagents. The primary antibody (TF antibody, TFRC antibody, TF antibody fragment, or TFRC antibody fragment or variants thereof) serves as an antigen which can bind more than one secondary antibody. The secondary antibodies form a “bridge” between the primary antibody and the complex formed by the detectable marker and developing reagent (for example, a horseradish peroxidase-antiperoxidase complex).

Any suitable detection system can be used in accordance with the methods and kits of the present invention. Such detection systems are widely used in immunofluorescence applications, and can be imaged using techniques including, but not limited to, flow cytometry, microscopy, Western blotting, and ELISAs. Suitable detection systems can employ conjugates of secondary antibodies, conjugates of colloidal gold, or conjugates of secondary proteins, in order to amplify the signal from a primary protein (in the context of the present invention, the primary protein signal being amplified is bound a TF or TFRC antibody, which can or cannot be labeled, for example with a protein such as biotin), which is in turn being used to detect a specific target (in the context of the present invention, the target is a TF or TFRC expression product).

Suitable secondary conjugates for use in the methods and kits of the present invention can include, but are not limited to, enzyme conjugates of a secondary antibody and an enzyme such as horseradish peroxidase or alkaline phosphatase; enzyme conjugates of avidin or streptavidin and an enzyme such as horseradish peroxidase or alkaline phosphatase; enzyme conjugates of protein A or protein G and an enzyme such as horseradish peroxidase or alkaline phosphatase; conjugates of colloidal gold and a secondary antibody; conjugates of colloidal gold and avidin or streptavidin; conjugates of magnetic particles and a secondary antibody; and conjugates of secondary antibodies and labels such as fluorescent dyes and biotin. The present invention is not limited to any particular detection systems, and it is considered within the ability of the person of ordinary skill in the art to utilize these or other detection systems in accordance with the present invention. These secondary conjugates (also referred to as labels in the context of the present invention) are useful for visualizing antigen-antibody complexes.

The antibody or antibody fragment of the present invention can also be adapted for utilization in an immunometric assay, also known as a “two-site” or “sandwich” assay. In a typical immunometric assay, a quantity of unlabeled antibody (or fragment of antibody), is bound to a solid support that is insoluble in the fluid being tested and a quantity of detectably labeled soluble antibody is added to permit detection and/or quantitation of the ternary complex formed between solid-phase antibody, antigen, and labeled antibody.

For purposes of in vivo imaging of breast, ovary, cervix, prostate, colon, stomach, kidney, liver, head, neck, lung, blood, pancreas, skin, testicle, thyroid and brain, most preferentially human breast, ovary, head, neck, brain, and prostate, in particular human prostate cancer and other cancers using the antibodies or antibody fragments of the present invention, there are many different labels and methods of labeling known to those of ordinary skill in the art. Examples of the types of labels which can be used in the present invention include radioactive isotopes, paramagnetic isotopes, and compounds which can be imaged by positron emission tomography (PET).

Pharmaceutical Compositions and Methods of Treatment

Another aspect of the invention provides a composition comprising any of these antibodies, optionally in combination with a pharmaceutically acceptable carrier. In another aspect, an antibody of the present invention is optionally in combination with one or more active agents, drugs, or hormones. In another embodiment, a humanized Alper-TF mAb is encompassed and is useful in the treatment of any disease or disorder characterized by aberrant TF or TFRC expression, including, for example, cancer, prostate cancer, or iron deficiency anemia.

The present invention also provides a method of treating human or animal subjects suffering from or at risk of a cancer that expresses TF and/or TFRC, such as cancer of the breast, ovary, cervix, prostate, colon, stomach, kidney, liver, head, neck, lung, blood, pancreas, skin, testicle, thyroid, brain, and prostate, most preferentially human prostate, the method comprising administering to the subject a therapeutically effective amount of an antibody of the present invention or a pharmaceutical composition comprising a therapeutically effective amount of an antibody of the present invention.

The present invention also provides a method of treating human or animal subjects suffering from or at risk of iron deficiency anemia.

The term “subject” as used herein refers to any subject in need of treatment, preferably a human patient or subject.

The term “therapeutically effective amount” as used herein refers to an amount of a therapeutic agent needed to treat, ameliorate, or prevent a targeted disease or condition, or to exhibit a detectable therapeutic or preventative effect. For any antibody, the therapeutically effective dose can be estimated initially either in cell culture assays or in animal models, usually in rodents, rabbits, dogs, pigs, or primates. The animal model can also be used to determine the appropriate concentration range and route of administration. Such information can then be used to determine useful doses and routes for administration in humans.

An effective amount for a human subject can depend upon the severity of the disease state; the general health of the subject; the age, weight and gender of the subject; diet; time and frequency of administration; drug combination(s); and reaction sensitivities, and tolerance/response to therapy and can be determined by routine experimentation and is within the judgment of the clinician. Generally, an effective dose will be from 0.01 mg/kg to 50 mg/kg, 0.1 mg/kg to 20 mg/kg, about 1 mg/kg to about 15 mg/kg.

Compositions can be administered individually to a patient or can be administered in combination with other agents, drugs, or hormones. According to some aspects, antibodies can be conjugated with these agents. A summary of the ways in which the antibodies of the present invention can be used therapeutically includes direct cytotoxicity by the antibody, either mediated by complement or by effector cells, or by conjugation to anti-tumor drugs, toxins, and radionuclides. Antibodies can also be used for ex vivo removal of tumor cells from the circulation or from bone marrow.

Cytotoxic proteins can include, but are not limited to, Ricin-A, Pseudomonas toxin, Diphtheria toxin, and tumor necrosis factor. Diagnostic radionuclides and cytotoxic agents such as cytotoxic radionuclides, drug and proteins can also be conjugated to the antibodies of the present invention. Examples of radionuclides which can be coupled to antibodies and selectively delivered in vivo to sites of antigen include ²¹²Bi, ¹³¹I, ¹⁸⁶Re, and ⁹⁰Y, among others. Radionuclides can exert their cytotoxic effect by locally irradiating the cells, leading to various intracellular lesions, as is known in the art of radiotherapy. Examples of cytotoxic drugs which can be conjugated to antibodies and subsequently used for in vivo therapy include, but are not limited to, daunorubicin, doxorubicin, methotrexate, and Mitomycin C. Cytotoxic drugs can interface with critical cellular processes including DNA, RNA, and protein synthesis.

A dose at which the antibody molecule of the present invention is administered depends on the nature of the condition to be treated, and on whether the antibody molecule is being used prophylactically or to treat an existing condition. If administered prophylactically (i.e., as a vaccine), the antibody is administered in an amount effective to elicit an immune response in the subject.

If the antibody molecule has a short half-life (e.g., 2 to 10 hours), it can be necessary to give one or more doses per day. Alternatively, if the antibody molecule has a long half-life (e.g., 2 to 15 days), it can only be necessary to give a dosage once per day, per week, or even every 1 or 2 months.

A pharmaceutical composition can also contain a pharmaceutically acceptable carrier for administration of the antibody. The carrier should not itself induce the production of antibodies harmful to the individual receiving the composition and should not be toxic. Suitable carriers include those known in the art, and can be selected from large, slowly metabolized macromolecules such as proteins, polypeptides, liposomes, polysaccharides, polylactic acids, polyglycolic acids, polymeric amino acids, amino acid copolymers, and inactive virus particles, although suitable carriers are not limited to these examples.

Preferred forms for administration include forms suitable for parenteral administration, e.g. by injection or infusion, for example by bolus injection or continuous infusion. Where the product is for injection or infusion, it can take the form of a suspension, solution, or emulsion in an oily or aqueous vehicle and it can contain formulatory agents, such as suspending, preservative, stabilizing, and/or dispersing agents. Alternatively, the antibody molecule can be in dry form, for reconstitution before use with an appropriate sterile liquid.

Once formulated, the compositions of the invention can be administered directly to the subject. The subjects to be treated can be animals. However, it is preferred that the compositions are adapted for administration to human subjects.

A pharmaceutical composition of this invention can be administered by any number of routes including, but not limited to, oral, intravenous, intramuscular, intra-arterial, intramedullary, intrathecal, intraventricular, transdermal, transcutaneous, subcutaneous, intraperitoneal, intranasal, enteral, topical, sublingual, intravaginal, or rectal routes. Hyposprays can also be used to administer the pharmaceutical compositions of the invention. Therapeutic compositions can be prepared as injectables, either as liquid solutions or suspensions. Solid forms suitable for solution in, or suspension in, liquid vehicles prior to injection can also be prepared.

Direct delivery of the compositions will generally be accomplished by injection, subcutaneously, intraperitoneally, intravenously, or intramuscularly, or delivered to the interstitial space of a tissue. Dosage treatment can be a single dose schedule or a multiple dose schedule.

When a TF antibody, TFRC antibody, TF antibody fragment, or TFRC antibody fragment composition is to be administered by a route using the gastrointestinal tract, the composition can contain additional agents which protect the antibody from degradation but which release the antibody once it has been absorbed from the gastrointestinal tract. Such additional agents are known to those skilled in the art.

Antibodies of the present invention can also be administered in methods of conducting gene therapy. In order to achieve this, DNA sequences encoding the heavy and light chains of the antibody molecule under the control of appropriate DNA components are introduced into a patient such that the antibody chains are expressed from the DNA sequences and assembled in situ.

In yet another aspect, the present invention relates to a method of treating cancer by administering an effective amount of an antibody or antibody fragment disclosed herein that binds to TF or TFRC. In some embodiments, the antibody or antibody fragment is sufficient to reduce growth of cancerous cells. In certain embodiments, the cancer is prostate cancer.

In further embodiments, the cancerous cells are selected from the group of solid tumors including but not limited to breast cancer, colon cancer, prostate cancer, lung cancer, sarcoma, renal metastatic cancer, thyroid metastatic cancer, and clear cell carcinoma.

In a further aspect, the present invention relates to a method of delaying development of metastasis in a subject suffering from cancer comprising administering an effective amount of a composition comprising at least anti-TF and anti-TFRC antibodies and antibody fragments described herein.

In another aspect, the present invention relates to a method of inhibiting growth and/or proliferation of cancer cells in vitro or in a subject comprising administering an effective amount of a composition comprising at least anti-TF and anti-TFRC antibodies and antibody fragments described herein, associated with (including linked to) a chemotherapeutic agent, to the cell culture or sample, or to the subject.

In some aspects, the present invention relates to a method of delivering a therapeutic agent to a cancerous cell in a subject by administering to the subject an effective amount of a composition comprising at least anti-TF and anti-TFRC antibodies and antibody fragments described herein. In some embodiments, the antibody or antibody fragment is delivered to the subject in combination with (including linked to) another therapeutic agent.

In some embodiments, the antibodies of the present invention are delivered to a subject in need thereof intravenously, intramuscularly, intraarterially, intrathecally, intracapsularly, intraorbitally, intracardiacally, intradermally, intraperitoneally, transtracheally, subcutaneously, subcuticularly, intraarticularly, subcapsularly, subarachnoidally, intraspinally, epidurally, and intrasternally.

TF Expression Products as Drug Development Targets

In addition, the present invention relates to the molecular mechanisms resulting in TF or TFRC antigens for Alper-TF mAb, such as precursor TF or TFRC being secreted by cancer cells, such as prostate cancer cells. This expression of TF or TFRC antigens presents novel drug development targets, and accordingly, the present invention also relates to the use of such TF and TFRC antigens as biomarkers that can be targeted not only by the TF or TFRC antibodies or antibody fragments of the present invention, but also by various other molecules, such as siRNA, antisense oligonucleotides, vaccines, and chemical compounds.

Methods for developing drugs useful in treating and/or diagnosing diseases characterized by the expression of TF or TFRC antigens for Alper-TF mAb can include the steps of identifying TF and TFRC antigens for Alper-TF mAb in a subject having a disease, such as prostate cancer, and utilizing those mechanisms of producing TF and TFRC antigens for Alper-TF mAb to develop and identify drugs that specifically target those molecular mechanisms.

Once candidate drugs have been developed based on the TF and TFRC antigens, the TF and TFRC antigens and TF antibodies, TFRC antibodies, TF antibody fragments, and TFRC antibody fragments of the present invention can be used to aid in screening the various drug candidates, in order to identify those drug candidates that exhibit a desired level of specificity for diseased cells presenting TF or TFRC expression products.

Targeting TF and TFRC-Expressing Cells

In one aspect, the present invention relates to the use of anti-TF and anti-TFRC antibodies and antibody fragments described herein to target a payload to a TF- and TFRC-expressing cell or to a tissue or other structure associated with TF and/or TFRC. In some embodiments, the payload is a therapeutic agent. In various embodiments, the payload comprises at least an anti-cancer therapeutic. In other embodiments, the payload is a microbe, such as a bacterium or virus. For example, the antibodies can be attached to a virus or virus like particle that can deliver an exogenous gene (e.g., for gene therapy) or to a liposome, e.g., a liposome that encapsulates a therapeutic agent or exogenous gene. An exemplary method for using an antibody to target a virus is described in Roux et al. (1989) Proc Natl Acad Sci USA (1989) 86: 9079-9083. See also Curr Gene Ther. (2005) 5: 63-70 and Hum Gene Ther. (2004) 15:1034-1044.

The anti-TF and anti-TFRC antibodies or antibody fragments of the present invention may also be attached to liposomes containing a therapeutic agent such as chemotherapeutic agents. Attachment of antibodies to liposomes may be accomplished by any known cross-linking agent such as heterobifunctional cross-linking agents that have been widely used to couple toxins or chemotherapeutic agents to antibodies for targeted delivery. For example, conjugation to liposomes can be accomplished using the carbohydrate-directed cross-linking reagent 4-(4-maleimidophenyl) butyric acid hydrazide (MPBH). See Duzgunes et al. (1992) J. Cell. Biochem. Abst. Suppl. 16E 77. Liposomes containing antibodies can also be prepared by well-known methods See DE Pat. No. 3,218,121; Epstein et al. (1985) Proc. Natl. Acad. Sci. USA, 82: 3688-92; Hwang et al. (1980) Proc. Natl. Acad. Sci. USA, 77: 4030-34; U.S. Pat. Nos. 4,485,045 and 4,544,545.

The anti-TF and anti-TFRC antibodies or antibody fragments of the present invention may also be attached to labels that can be used to detect tumors in vivo. For example, the antibody can be labeled with an MRI detectable label or a radiolabel. The subject can be evaluated using a means for detecting the detectable label. For example, the subject can be scanned to evaluate localization of the antibody within the subject. For example, the subject is imaged by NMR or other tomographic means.

Examples of labels useful for diagnostic imaging include radiolabels such as ³¹¹I, ¹¹¹In, ¹²³I, ⁹⁹mTc, ³²P, ³³P, ¹²⁵I, ³H, ¹⁴C, and ¹⁸⁸Rh; fluorescent labels such as fluorescein and rhodamine; nuclear magnetic resonance active labels; positron emitting isotopes detectable by a positron emission tomography (“PET”) scanner; chemiluminescent labels such as luciferin; and enzymatic markers such as peroxidase or phosphatase. Short range radiation emitters, such as isotopes detectable by short range detector probes, can also be employed. The anti-TF and anti-TFRC antibodies or antibody fragments of the present invention can be labeled with such reagents using known techniques. See Wensel and Meares (1983) Radioimmunoimaging and Radioimmunotherapy, Elsevier, New York for techniques relating to the radiolabeling of antibodies and Colcher et al. (1986) Meth. Enzymol. 121: 802 816.

In some embodiments, the subject can be “imaged” in vivo using known techniques such as radionuclear scanning using a gamma camera or emission tomography. See A. R. Bradwell et al., “Developments in Antibody Imaging”, Monoclonal Antibodies for Cancer Detection and Therapy, R. W. Baldwin et al., (eds.), pp. 65-85 (Academic Press 1985). Alternatively, a positron emission transaxial tomography scanner, such as designated Pet VI located at Brookhaven National Laboratory, can be used where the radiolabel emits positrons (e.g., ¹¹C, ¹⁸F, 15O, and ¹³N).

Magnetic Resonance Imaging (MRI) uses NMR to visualize internal features of living subject, and is useful for prognosis, diagnosis, treatment, and surgery. MRI can be used without radioactive tracer compounds for obvious benefit. Some MRI techniques are summarized in EP0 502 814 A. Generally, the differences related to relaxation time constants T1 and T2 of water protons in different environments are used to generate an image. However, these differences can be insufficient to provide sharp high resolution images.

In other embodiments, the anti-TF and anti-TFRC antibodies or antibody fragments can also be labeled with an indicating group containing the NMR active ¹⁹F atom, or a plurality of such atoms inasmuch as (i) substantially all of naturally abundant fluorine atoms are the ¹⁹F isotope and, thus, substantially all fluorine containing compounds are NMR active; (ii) many chemically active polyfluorinated compounds such as trifluoracetic anhydride are commercially available at relatively low cost, and (iii) many fluorinated compounds have been found medically acceptable for use in humans such as the perfluorinated polyethers utilized to carry oxygen as hemoglobin replacements. After permitting such time for incubation, a whole body MRI is carried out using an apparatus such as one of those described by Pykett (1982) Scientific American, 246: 78-88 to locate and image EGFR distribution.

The anti-TF and anti-TFRC antibodies or antibody fragments of the present invention may also be attached to therapeutic agents (i.e., agents that treat, ameliorate the symptoms of, or prevent disease or recurrence of disease). In some embodiments, the therapeutic agent is an anti-tumor drug, such as a cytotoxic and/or chemotherapeutic agent, toxin or radionuclide. Examples of cytotoxic and chemotherapeutic agents include, but are not limited to, taxol, cytochalasin B, gramicidin D, vinblastine, doxorubicin, daunorubicin, a maytansinoid (e.g., maytansinol or the DM1 maytansinoid, a sulfhydryl-containing derivative of maytansine), methotrexate, mitoxantrone, mithramycin, actinomycin D, Mitomycin C, 1-dehydrotestosterone, glucocorticoids, procaine, taxane, tetracaine, lidocaine, propranolol, and puromycin and analogs or homologs thereof. Cytotoxic proteins can include, but are not limited to, Ricin-A, Pseudomonas toxin, Diphtheria toxin, and tumor necrosis factor.

Diagnostic radionuclides and cytotoxic agents such as cytotoxic radionuclides, drug and proteins can also be conjugated to the antibodies or antibody fragments of the present invention. Examples of radionuclides which can be coupled to antibodies and selectively delivered in vivo to sites of antigen expression include, but are not limited to, ²¹²Bi, ¹³¹I, ¹⁸Re, and ⁹⁰Y, among others. Radionuclides can exert their cytotoxic effect by locally irradiating the cells, leading to various intracellular lesions, as is known in the art of radiotherapy.

In other aspects, the present invention relates to a method for delivering a neuropharmaceutical or diagnostic agent across the blood brain barrier to the brain of a subject in need thereof. In some embodiments, the method comprises administering to the subject in need thereof a therapeutically effective amount of a composition comprising anti-TF and anti-TFRC antibodies or antibody fragments of the present invention associated with (including linked to) neuropharmaceutical or diagnostic agents.

In some embodiments, the subject can have or be at risk for a neurological disorder.

The neuropharmaceutical agent can be an agent having a therapeutic or prophylactic effect on a neurological disorder or any condition which affects biological functioning of the central nervous system.

Examples of neurological disorders include, but are not limited to, cancer (e.g. brain tumors), Autoimmune Deficiency Syndrome (AIDS), stroke, epilepsy, Parkinson's disease, multiple sclerosis, neurodegenerative disease, trauma, depression, Alzheimer's disease, migraine, pain, or a seizure disorder.

Classes of neuropharmaceutical agents which can be used include, but are not limited to, proteins, antibiotics, adrenergic agents, anticonvulsants, small molecules, nucleotide analogs, chemotherapeutic agents, anti-trauma agents, peptides and other classes of agents used to treat or prevent a neurological disorder. Examples of proteins include, but are not limited to, CD4 (including soluble portions thereof), growth factors (e.g. nerve growth factor and interferon), dopamine decarboxylase and tricosanthin. Examples of antibiotics include, but are not limited to, amphotericin B, gentamycin sulfate, and pyrimethamine. Examples of adrenergic agents (including blockers) include, but are not limited to, dopamine and atenolol. Examples of chemotherapeutic agents include, but are not limited to, adriamycin, methotrexate, cyclophosphamide, etoposide, and carboplatin. An example of an anticonvulsant which can be used is valproate and an anti-trauma agent which can be used is superoxide dismutase. Examples of peptides include, but are not limited to, somatostatin analogues and enkephalinase inhibitors. Examples of nucleotide analogs include, but are not limited to, azido thymidine (hereinafter AZT), dideoxy Inosine (ddI) and dideoxy cytodine (ddc).

In some embodiments, the anti-TF and anti-TFRC antibodies or antibody fragments of the present invention are associated with a diagnostic agent which may be an antibody or antibody fragment. For example, the diagnostic agent can be an antibody to amyloid plaques. For example, when conjugated to anti-TF and anti-TFRC antibodies or antibody fragments of the present invention, this diagnostic agent antibody can be transferred across the blood brain barrier and can then subsequently immunoreact with amyloid plaques. Such an immunoreaction is indicative of Alzheimer's Disease.

The following examples are non-limiting illustrative examples.

### EXAMPLES

### Example 1

Approximately 1 μg of purified Alper-TF mAb was suspended in PBS and applied under reducing conditions (boiled 3 minutes in sample buffer with beta-mercaptoethanol and 10% SDS) to 10% Bis-Tris gel. The gel was run at 120 volts, and then stained with Coomassie Blue (0.1% (w/v) Coomassie blue R350, 20% (v/v) methanol, and 10% (v/v) acetic acid), destained in 50% (v/v) methanol in water with 10% (v/v) acetic acid.

Under denatured conditions, the heavy chain of Alper-TF mAb was detected at ˜50 kDa. The light chain of Alper-TF mAb was detected at ˜25 kDa.

### Example 2

A volume of 20 μl of sample buffer containing 50 μg of purified recombinant TF in sample buffer were boiled 90° C. for 3 minutes and loaded into an 8% Tris-Glycine gel, along with 15 μl of molecular markers. The gel was run at 125 volts for 1.5 hours. The gel was then transferred to a PVDF membrane. The membrane was incubated with Alper-TF mAb at 4° C. overnight. Subsequently, the membrane was rinsed 3 times for 10 minutes in TBST, incubated with secondary antibody (Sheep anti-mouse IgG-HRP, [Cat#Na931V Lot #352104, GE Healthcare] 1:1,000 diluted in 2% NFDM in TBST) for one hour, rinsed 3 times for 10 minutes in TBST, treated with ECL, and exposed to x-ray film.

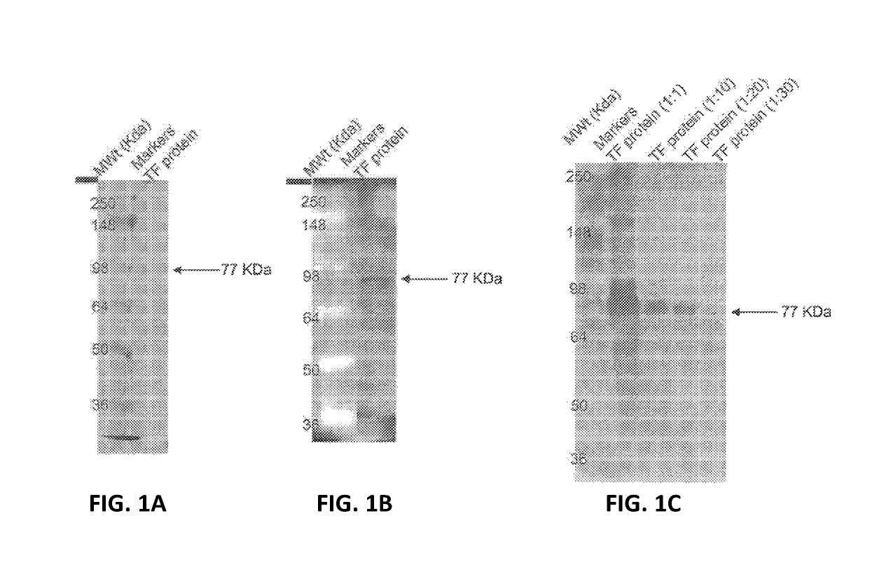

The experiment was repeated at least three times, and FIGS. 1A, 1B, and 1C are representative images. FIG. 1A is a Commassie Blue staining of the gel showing a single band at about 77 kDa. FIG. 1B shows one representative image of the results of the Western Blot using Alper-TF mAb. FIG. 1C shows another representative image of the results of the Western Blot using Alper-TF mAb. In addition to the 50 μg of purified TF, 50 μg of purified TF that was serially diluted 1:1, 1:10, 1:20, or 1:30 (v:v) was also loaded into the gel, as indicated by the labeling of the lanes. Alper-TF mAb recognizes a 77 kDa protein (TF).

### Example 3

The volume of 20 μl of sample buffer containing 50 μg of purified recombinant TFRI (also known as TFRC) (Company Origene; Catalog tp326147), another purified recombinant TFRI (also known as TFRC (Company Origene; Catalog tp300980), purified recombinant transferrin receptor 2 (also known as “TFRII”) (Company Origene; Catalog TP320060), and purified recombinant TF (Origene Technologies, Inc.; Catalog TP309184) in sample buffer were boiled 90° C. for 3 minutes and loaded into an 10% Tris-Glycine gel, along with 15 μl of molecular markers. The gel was run at 120 volts for 1.5 hours. The gel was then transferred to a PVDF membrane. The membrane was incubated with Alper-TF mAb at 4° C. overnight. Subsequently, the membrane was rinsed 3 times for 10 minutes in PBST, incubated with secondary antibody (Sheep anti-mouse IgG-HRP, [Cat#Na931V Lot #352104, GE Healthcare]1:1,000 diluted in 2% NFDM in PBST) for one hour, rinsed 3 times for 10 minutes in PBST, treated with ECL, and exposed to x-ray film.

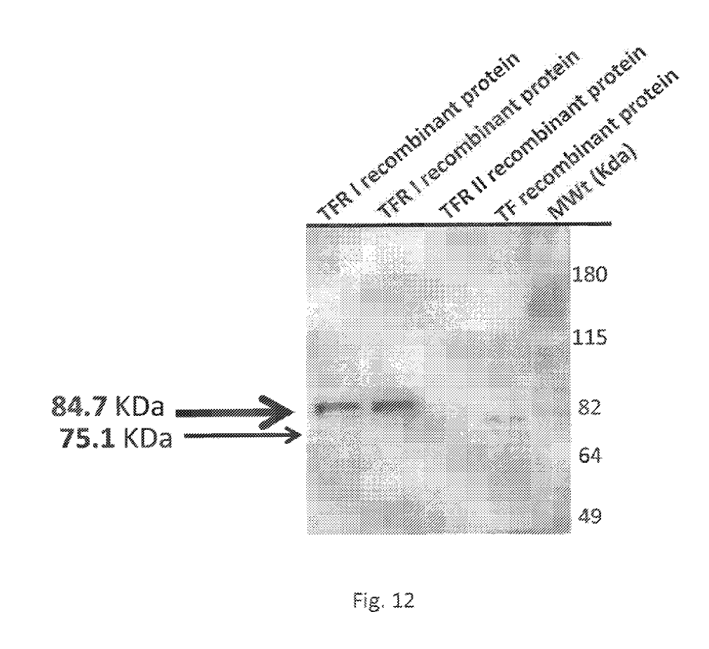

The experiment was repeated at least three times, and FIG. 12 is a representative image. Alper-TF mAb recognizes a ˜75 kDa protein (TF) and a ˜84.7 KDa protein (TFRC) but not transferrin receptor protein 2.

### Example 4

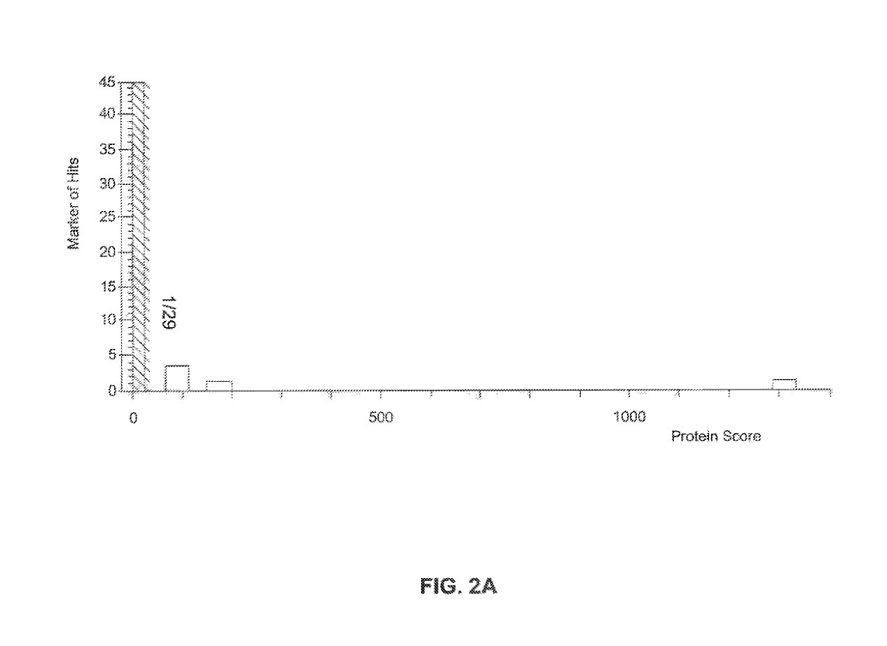

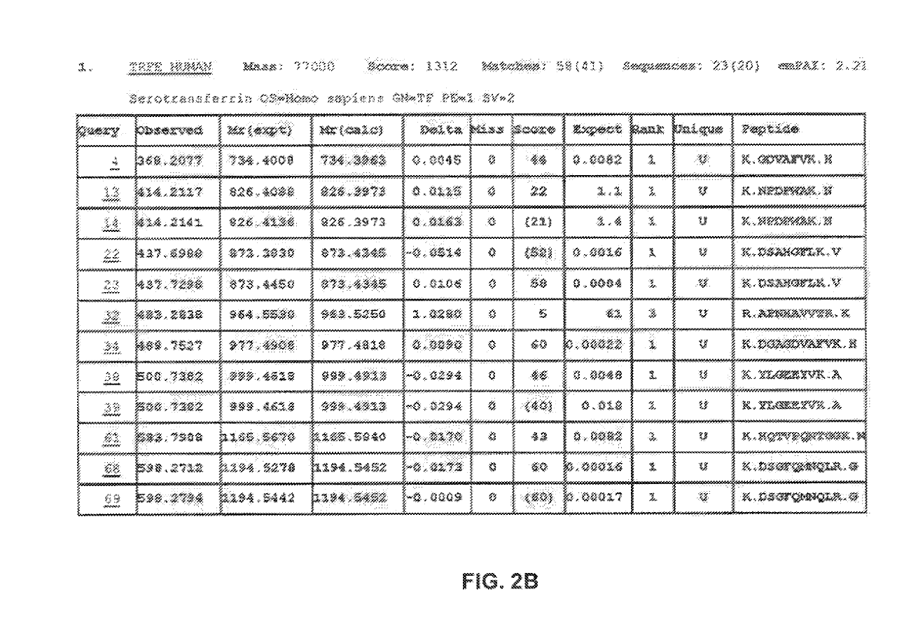

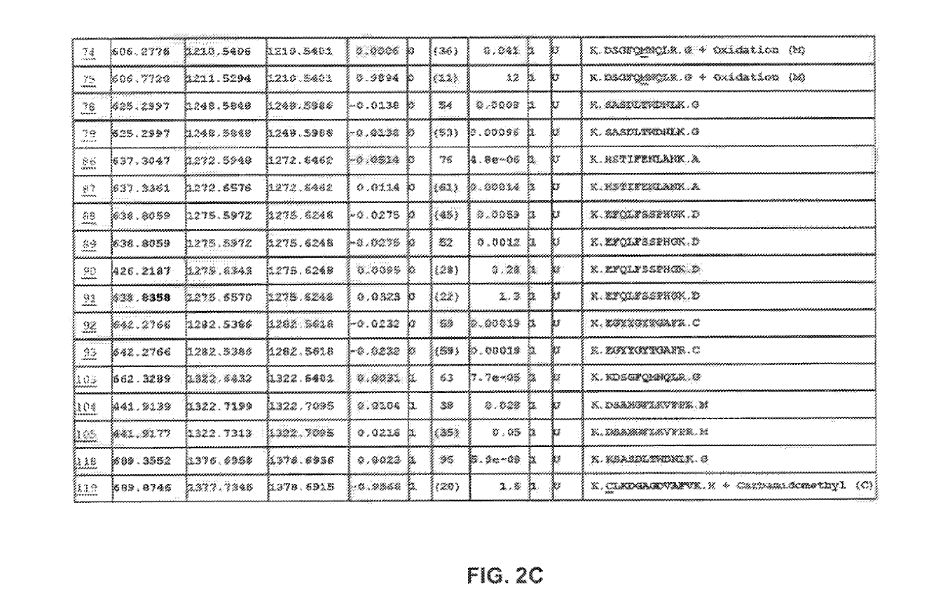

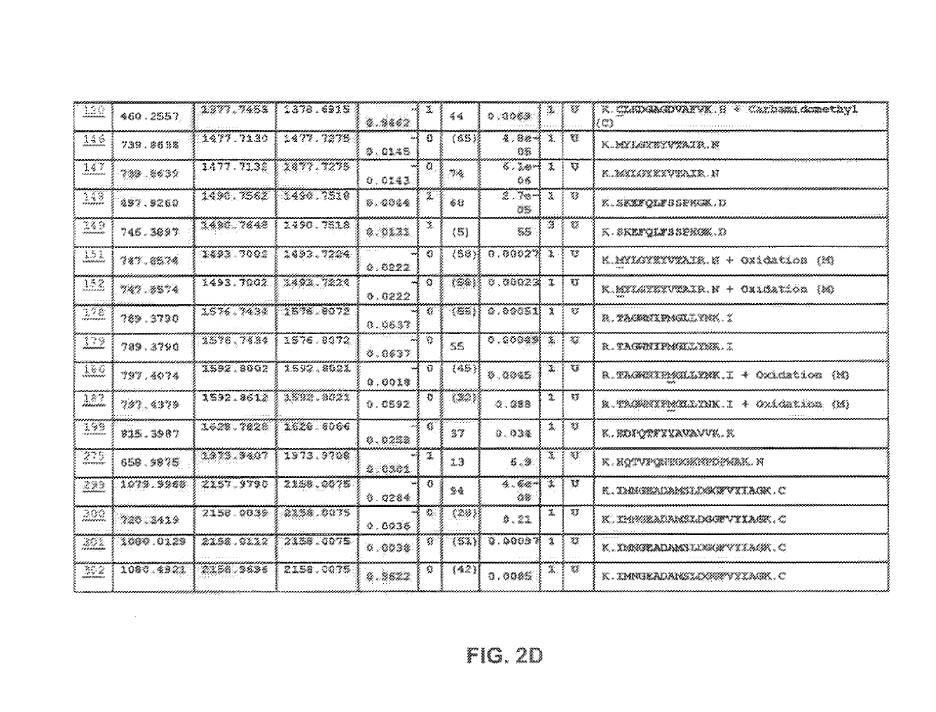

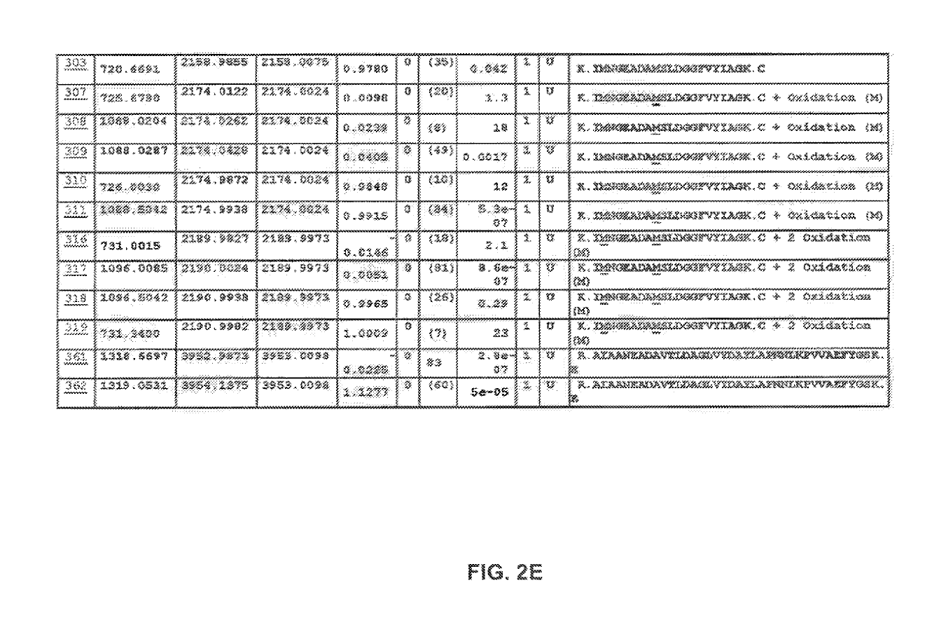

The antigen for Alper-TF mAb was isolated, digested with trypsin, and analyzed by MALDI-MS. The Mascot protein database was searched using the mass spectrometry data. The search identified the antigen as corresponding to the human serotransferrin protein (SwissProt TRFE_HUMAN, P02787-1), identifying correspondence to 58 partial TF sequences contained in the database. Protein scores were derived from an ions score as a non-probabilistic basis for ranking protein hits. Based on the probability based mowse scoring, ions score is −10*Log(P), where P is the probability that the observed match is a random event. Individual ions scores >34 indicate identity or extensive homology (p<0.05). FIG. 2A depicts the ions score graphically. The search also identified an albumin (fragment) and hemoglobin alpha and beta, likely contaminants. FIGS. 2B-2E show the details of the 58 matches identified by this search.

### Example 5

The protein concentrations of OPCT1 cell culture supernatant were determined using BCA Assay (Smith et. al. Anal. Biochem. 150: 76-85, 1985, and Pierce Chemical Co., Rockford, Ill.). OPCT1 cells were derived from prostate tumor epithelium (T1cN0M0, Gleason 3+3) from patients who received no chemotherapy, no radiotherapy, and no hormone treatment (cells were purchased from Asterand Inc.). The samples were then lyophilized, redissolved to 4 mg/ml in SDS Boiling Buffer, and heated in a boiling water bath for 3 minutes before loading onto an 8% acrylamide slab gel.

Western Blotting Methods: 8% acrylamide slab gel electrophoresis was carried out about 4 hours at 15 mA/gel. After slab gel electrophoresis, the gel was placed in transfer buffer (12.5 mM Tris, pH 8.8, 96 mM Glycine, 20% MeOH) and transferred onto a PVDF membrane overnight at 200 mA and approximately 100 volts/2 gels. The following proteins (Sigma Chemical Co., St. Louis, Mo.) were used as molecular weight standards: myosin (220,000), phosphorylase A (94,000), catalase (60,000), actin (43,000), carbonic anhydrase (29,000) and lysozyme (14,000). The blots were then blocked for two hours in 5% nonfat dry milk (NFDM) in Tween-20 tris buffer saline (TTBS) and rinsed in TTBS. The membrane was incubated in primary antibody (Alper-TF mAb antibody diluted 1:125 in 2% NFDM TTBS) overnight. The membrane was rinsed 3 times for 10 minutes in TTBS, incubated with secondary antibody (Sheep anti-mouse IgG-HRP, [Cat#Na931V Lot #352104, GE Healthcare]1:1,000 diluted in 2% NFDM in TTBS) for two hours, rinsed 3 times for 10 minutes in TTBS, treated with ECL, and exposed to x-ray film.

A protein with a 77 kDa MWt was detected in culture supernatant prepared from OCPT1 cells when probed with Alper-TF mAb, demonstrating that the Alper-TF mAb specifically binds to TF from human prostate cancer cells in a Western Blot application.

### Example 6

Plasma samples from healthy control and prostate cancer patients, as determined by pathology of patient biopsies, were assayed for levels of antigen by ELISA using Alper-TF mAb. Plasma samples were diluted with PBS at a ratio of 1:100 and coated onto polysorp ELISA plates (Nalgene NUNC® International, Rochester, N.Y.) at 100 μl/well and incubated at 4° C. overnight. The plasma samples were analyzed in a blinded fashion. Wells were washed with PBS and incubated at room temperature for one hour with blocking buffer (5% BSA in PBS). After washing with PBS, the primary antibody, Alper-TF mAb, was added in dilution buffer (45 μg/ml) (PBS buffer, 1% BSA, 0.01% Tween-20). The wells were washed with PBS/0.03% Tween-20 and incubated at room temperature for one hour with 100 μl/well secondary antibody (HRP-Donkey anti-mouse IgG, Jackson ImmunoResearch, West Grove, Pa.) diluted 1:3000. After washing the wells, 100 μl Immunopure TMB substrate solution (Pierce, Rockford, Ill.) was added. The color reaction was stopped by the addition of 100 μl/well 1 M H₂SO₄. Analysis was performed with a Multiscan Plus ELISA Reader (Thermo Electron Inc.).

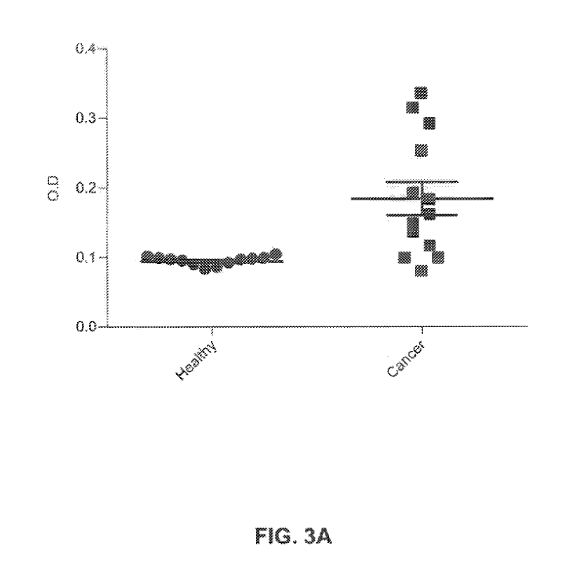

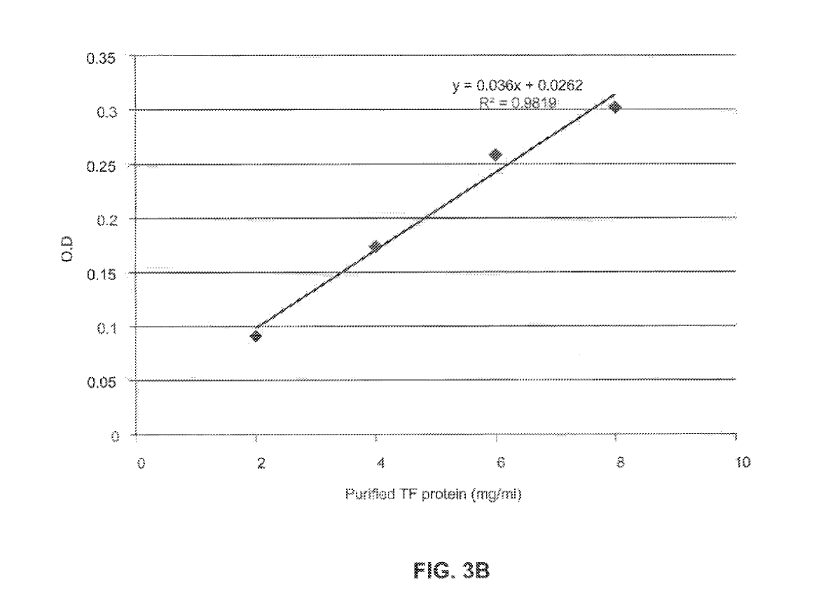

FIG. 3A shows the optical density (OD) values of antigen levels in healthy and prostate cancer patients. The results show a significant increase in antigen levels in cancer patients as compared to healthy controls (p<0.01 as determined by unpaired T-test). Alper-TF mAb is useful in immunoassays for the detection of prostate cancer. FIG. 3B shows the linear correlation between the concentration of purified TF protein and absorbance values (OD), used as a standard curve in this assay.

### Example 7

In this example, normal prostate cancer cells from cell line OPCN1 were used together with early-stage prostate cancer cells from cell line OPCT1, and with late-stage metastatic prostate cancer cells from cell line LNCaP. OPCN1 and OPCT1 cell lines were derived from normal prostate tissue and cancerous prostate tissue from the same tumor after prostatectomy. See Palazzolo et al. (2005) Proc. Amer. Assoc. Cancer Res. 46: Abstract 1974; also available from Asterand Inc. LNCaP cells are available from ATCC, #CRL-1740. Cells from each cell line were prepared, fixed by incubation with methanol, and incubated overnight with Alper-TF mAb in a standard indirect-immunofluorescent staining assay. See, for example, Ausubel et al., supra

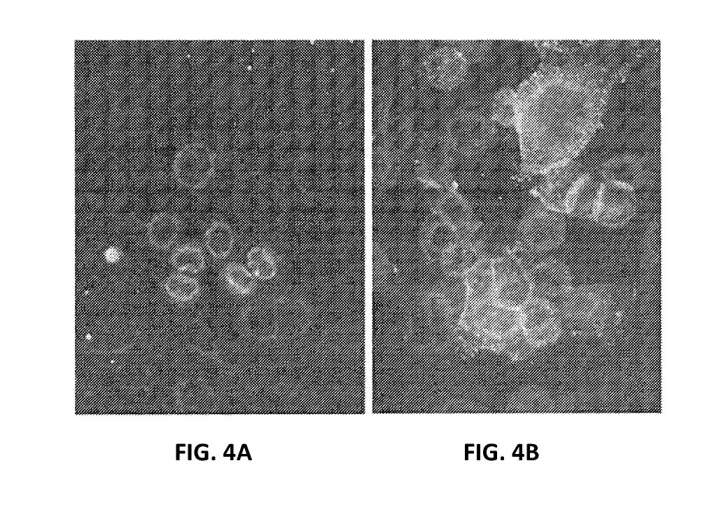

FIG. 4A shows one representative image of the results of the indirect-immunofluorescent assay using the Alper-TF mAb with normal prostate cells (OPCN1). FIG. 4B shows one representative image of the results of the indirect-immunofluorescent staining assay using Alper-TF mAb with early-stage prostate cancer cells (OPCT1). Non-cancerous prostate cells show non-punctate staining, whereas early-stage prostate cancer cells show punctuate staining that is typical of localization to early endosomes. Alper-TF mAb is useful in immunocytochemical assays to detect prostate cancer. In one embodiment Alper-TF mAb is useful in immunocytochemical assays for the early detection of prostate cancer.

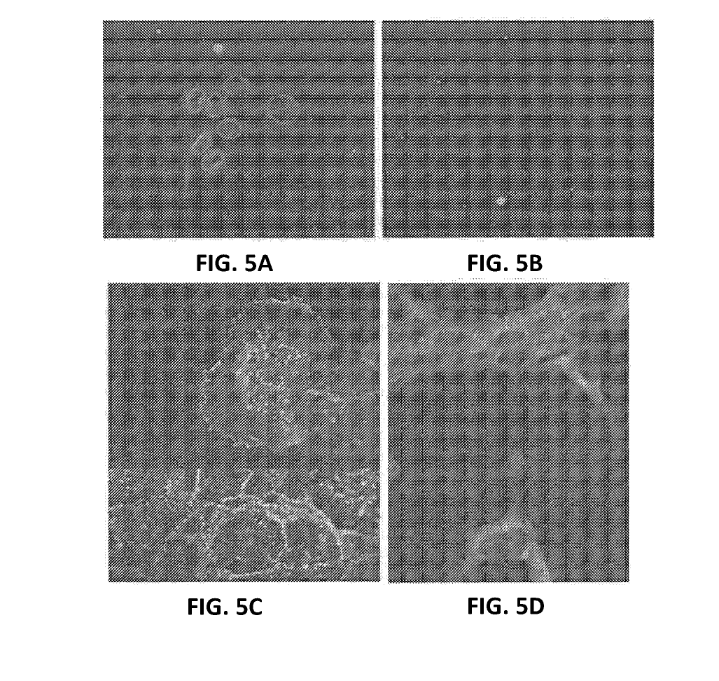

FIGS. 5A and 5B show two representative images of the results of an indirect-immunofluorescent staining assay using the Alper-TF mAb with normal prostate cells (OPCN1). Staining is non-punctate. FIG. 5C shows two representative image of the results of the indirect-immunofluorescent staining assay using the Alper-TF mAb with early-stage prostate cancer cells (OPCT1). FIG. 5C shows punctate staining typical of localization to early endosomes, and is distinguished from the non-punctate staining shown for non-cancerous prostate cells in FIGS. 5A and 5B. FIG. 5D shows one representative image of the results of an indirect-immunofluorescent staining assay using the Alper-TF mAb and late-stage prostate cancer cells (LNCaP). FIG. 5D shows non-punctate, diffuse staining that is distinguished from the punctate staining shown for early-stage cancerous prostate cells in FIGS. 5C and 4B, and the non-punctate staining that is shown for the non-cancerous prostate cells of FIGS. 4A, 5A, and 5B. Alper-TF mAb is useful in immunocytochemical assays to detect prostate cancer. In one embodiment, Alper-TF mAb is useful in immunocytochemical assays for distinguishing early and late stage prostate cancer, and for the early detection of prostate cancer.

### Example 8

A proprietary nanochip in conjunction with a carbon nanotube field-effect transistor (CNT-FET) platform (available from Fuzbien Technology Institute, Inc.) was used to compare Alper-TF mAb binding to antigen in blood from subjects with early-stage and late-stage prostate cancer to those of blood from healthy, age-matched subjects. The CNT-FET platform detects binding of a ligand, such as binding of an antigen to an antibody, using electronic detectors rather than conventional optical detectors. For each sample, 1 μl of blood was applied to the nanochip. Results indicated significant binding of Alper-TF mAb to its ligand in blood from subjects with prostate cancer, as compared to blood from healthy control subjects. There was no increased binding in blood from healthy, age-matched control subjects. In addition, patients with invasive prostate cancer demonstrated increased binding when compared to early-stage prostate cancer patients. Moreover, identification of samples from patients with prostate cancer using Alper-TF mAb was greater and more consistent than binding of commercial antibodies that target PSA and PMSA (currently recognized prostate cancer biomarkers), indicating utilization of Alper-TF mAb to detect antigen as a prostate cancer biomarker is superior to detection of PSA.

### Example 9

OPCT1 cells were cultured on glass-bottomed wells for 18 hours. The cells were permeabilized with Triton X-100. Transferrin from human serum conjugated to Texas Red (TxR-TF; Molecular Probes, Invitrogen) was added to the culture medium and incubated for 10 minutes. Cells were washed and fixed with 10% paraformaldehyde diluted in PBS for 15 minutes. Cells were again washed with PBS and subsequently incubated with Alper-TF mAb (5 μg/ml) for 15 minutes. Cells were again washed with PBS and subsequently incubated with FITC-conjugated mouse IgG (4 μg/ml) for 15 minutes. Cells were then visualized using an immunofluorescence microscope.

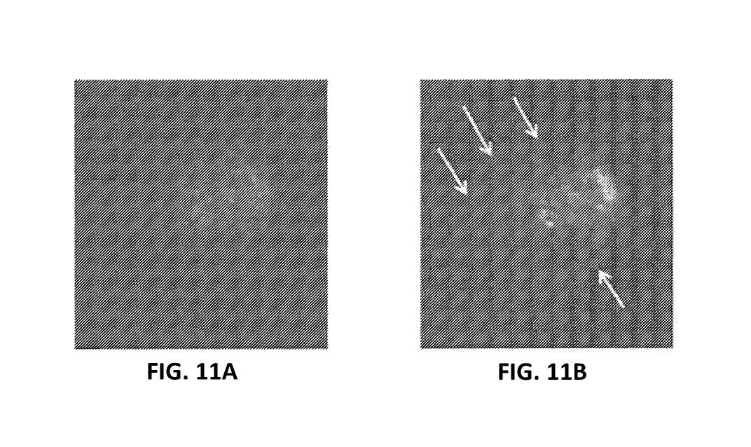

FIG. 11A shows a representative image of the results of the direct immunofluorescence assay for TxR-TF. As expected, TxR-TF, a known endosomal marker, was incorporated into the endosomes during the 10-minute incubation, as demonstrated by the punctate staining. FIG. 11B shows a representative image of the results of the Indirect immunofluorescence assay for FITC-labeled Alper-TF mAb. Alper-TF mAb fluorescence co-localized with all TxR-TF fluorescence in a similar punctate manner. This image confirms that Alper-TF mAb specifically binds TF, including exogenous TxR-TF localized to the endosomes.

### Example 10

Plasma samples were obtained from Asterand Plc. Plasma samples were from healthy control (n=15) and prostate cancer patients having low grade/stage cancer (stage I and II; n=9), and high grade/stage cancer (stage III and IV; n=4), as determined by pathology of patient biopsies. The patients were untreated. Samples were assayed for levels of antigen by ELISA using Alper-TF mAb, MAB2472 (R&D systems), sc-51829 (Santa Cruz), and ab38171 (Abcam). Each sample was repeated eight times. Plasma samples were diluted with PBS at a ratio of 1:100 and coated onto polysorp ELISA plates (Nalgene NUNC® International, Rochester, N.Y.) at 100 μl/well and incubated at 4° C. overnight. The plasma samples were analyzed in a blinded fashion. Wells were washed with PBS and incubated at room temperature for one hour with blocking buffer (5% BSA in PBS). After washing with PBS, the primary antibody: Alper-TF mAb. MAB2472 (R&D systems), sc-51829 (Santa Cruz), or ab38171 (Abcam), was added in dilution buffer (45 μg/ml) (PBS buffer, 1% BSA, 0.01% Tween-20). The wells were washed with PBS/0.03% Tween-20 and incubated at room temperature for one hour with 100 μl/well secondary antibody (HRP-Donkey anti-mouse IgG, Jackson ImmunoResearch, West Grove, Pa.) diluted 1:3000. After washing the wells, 100 μl Immunopure TMB substrate solution (Pierce, Rockford, Ill.) was added. The color reaction was stopped by the addition of 100 μl/well 1N H₂SO₄. Analysis was performed with a Multiscan Plus ELISA Reader (Thermo Electron Inc.).

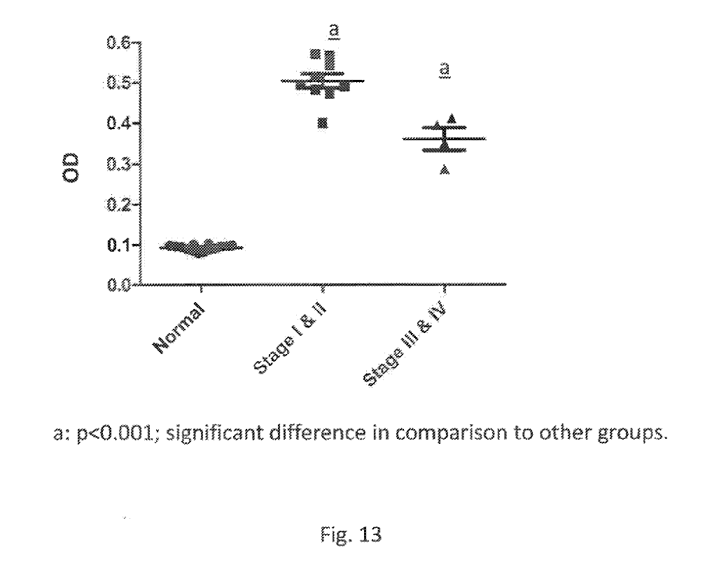

FIG. 13 shows the results of the assay using Alper TF mAb. Optical density (OD) values of antigen levels in i) healthy, ii) stage I and stage II prostate cancer patients, and iii) stage III and IV prostate cancer patients were plotted. The results show a significant increase in antigen levels in both groups of prostate cancer patients as compared to healthy controls (p<0.001 as determined by unpaired T-test). The results also show that Alper-TF mAb detects higher levels of antigen in low grade prostate cancers as compared to high grade cancers. Alper-TF mAb is thus useful in immunoassays for the detection and diagnosis of prostate cancer. Alper-TF mAb is also useful in immunoassays for the detection and diagnosis of stage I, stage II, stage III, and stage IV prostate cancer. Typically, a stage I prostate cancer is characterized by the presence of a tumor in less than about 5% of prostate tissue; stage II is characterized by tumor and elevated PSA levels; stage III is characterized by the tumor having spread through the prostatic capsule, and stage IV by the tumor having invaded other nearby structures.

Note that primary antibodies MAB2472 (R&D systems), sc-51829 (Santa Cruz), and ab38171 (Abcam), which are commercially available for the detection of TFRC, did not detect any antigen in healthy or prostate cancer patients.

1. An isolated antibody or antigen binding fragment thereof specific for transferrin receptor 1 (TFRC), comprising:
a heavy chain variable domain comprising at least one complementarity determining region (CDR) selected from SEQ ID NO: 2, SEQ ID NO: 3, and SEQ ID NO: 4, and
a light chain variable domain comprising at least one CDR selected from SEQ ID NO: 6, SEQ ID NO: 7, and SEQ ID NO: 8.

2. The isolated antibody or antigen binding fragment thereof of claim 1, wherein:
the heavy chain comprises the amino acids of SEQ ID NO: 1, and
the light chain comprises the amino acids of SEQ ID NO: 5.

3. An isolated antibody or antigen binding fragment thereof of claim 1 that binds TFRC, wherein:
the antibody binds to the same epitope as an antibody comprising a heavy chain variable domain comprising the amino acids of SEQ ID NO: 1, and
a light chain variable domain comprising the amino acids of SEQ ID NO: 5.

4. The isolated antibody or antigen binding fragment thereof of claim 1, wherein:
TFRC is a soluble protein having a molecular weight of about 85 kilodaltons as measured by gradient polyacrylamide gel electrophoresis.

5. An isolated antibody or antigen binding fragment thereof specific for TFRC, comprising:
a heavy chain variable domain comprising three CDRs selected from SEQ ID NO: 2, SEQ ID NO: 3, and SEQ ID NO: 4, and
a light chain variable domain comprising three CDRs selected from SEQ ID NO: 8, SEQ ID NO: 7, and SEQ ID NO: 8.

6. An isolated antibody or antigen binding fragment thereof specific for TFRC, comprising:
a light chain variable domain including:
a) a CDR1 comprising SEQ ID NO: 6,
b) a CDR2 comprising SEQ ID NO: 7, and
c) a CDR3 comprising SEQ ID NO: 8;
and a heavy chain variable domain including:
a) a CDR1 comprising SEQ ID NO: 2,
b) a CDR2 comprising SEQ ID NO: 3, and
c) a CDR3 comprising SEQ ID NO: 4.

7. The isolated antibody or antigen binding fragment thereof of claim 1, wherein:
the antibody is one of humanized, glycosylated, phosphorylated and labeled;
the antibody recognizes at least one epitope selected from the group consisting of the amino acids of SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 18, SEQ ID NO: 17, SEQ ID NO: 18, SEQ ID NO; 19, SEQ ID NO: 20, SEQ ID NO: 21, SEQ ID NO: 22, SEQ ID NO: 23, SEQ ID NO: 24, and SEQ ID NO: 25, or fragments of these amino acids; and
the antibody is capable of:
binding to TFRC with a specific affinity of between 10⁻⁸ M and 10⁻¹¹ M;
binding to a precursor form of TFRC;
binding to various forms of TFRC including a soluble form; a membrane-bound form; a phosphorylated form; and a non-phosphorylated form;
binding to TRFC with an affinity of between 10⁻⁸ and 10⁻¹¹ M;
selectively modulating the activity of TFRC;

8. An isolated antibody or antigen binding fragment thereof comprising:
a light chain and a heavy chain, wherein the sequences of both the light chain and the heavy chain have conservative sequence modifications relative to the sequences of the light chain and the heavy chain of the antibody of claim 1.

9. The isolated antibody or antigen binding fragment thereof of claim 8, wherein:
the sequence of the light chain and the sequence heavy chain are at least 80%, at least 85%, at least 90%, at least 95%, or at least 98% identical to the sequence of the light chain and the sequence of the heavy chain, respectively, of the antibody.

10. (canceled)

11. (canceled)

12. (canceled)

13. (canceled)

14. (canceled)

15. (canceled)

16. (canceled)

17. (canceled)

18. The isolated antibody or antigen binding fragment thereof of claim 7, wherein:
the antibody is capable of selectively reducing the activity of TFRC.

19. (canceled)

20. The isolated antibody or antigen binding fragment thereof of claim 7, wherein:
the antibody is labeled fluorescently, with an enzyme, or with a radioisotope.

21. The isolated antibody or antigen binding fragment thereof of claim 1 bound to TFRC.

22. The isolated antibody or antigen binding fragment thereof of claim 1 bound to TFRC, and further bound to a solid support.

23. The isolated antibody or antigen binding fragment thereof of claim 1, wherein the antibody is conjugated to an agent selected from the group consisting of: a detectable label, a cytotoxic radionuclide, a cytotoxic drug, and a cytotoxic protein.

24. A pharmaceutical composition comprising:
the antibody or antigen binding fragment thereof of claim 1, and a pharmaceutically acceptable carrier and/or diluent.

25. The antibody or antigen binding fragment thereof of claim 1, for use as a drug or in the manufacture of medicament for prevention or treatment of TFRC-related disorders.

26. The antibody or antigen binding fragment thereof of claim 1, wherein the TFRC-related disorder is one of cancer, prostate cancer, and iron deficiency anemia.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. A device comprising the antibody of any of claim 1, wherein the device is suitable for contacting or administering the antibody by at least one mode selected from parenteral, intravenous, intramuscular, intra-arterial, intrathecal, intracapsular, intraorbital, intracardiac, intradermal, intraperitoneal, transtracheal, subcutaneous, subcuticular, intraarticular, subcapsular, subarachnoid, intraspinal, epidural, and intrasternal.

58. A method of treating or preventing the progression of a TFRC-related disorder in a subject, wherein the method comprises administering to the subject an effective amount of the antibody or antigen binding fragment thereof of any of claim 1.

59. A method of ameliorating at least one symptom associated with a TFRC-related disorder in a subject, wherein the method comprises administering to the subject an effective amount of at least one antibody or antigen binding fragment thereof of claim 1.

60. The method of claim 58, wherein:
the antibody or antibody fragment is administered intravenously, intramuscularly, intraarterially, intrathecally, intracapsularly, intraorbitally, intracardiacally, intradermally, intraperitoneally, transtracheally, subcutaneously, subcuticularly, intraarticularly, subcapsularly, subarachnoidally, intraspinally, epidurally, and intrasternally.

61. The method of any of claim 58, wherein:
the TFRC-related disorder is one of cancer, prostate cancer, and iron deficiency anemia.

62. The method of claim 61, wherein the TFRC-related disorder is prostate cancer.

63. The method of claim 62, wherein the TFRC-related disorder is one of early stage, stage I, and stage II prostate cancer.

64. A method of delivering at least one therapeutic agent into a TFRC-expressing cell comprising contacting a TFRC-expressing cell with an anti-TFRC antibody or antigen binding fragment thereof according to claim 1, conjugated to a payload.

65. The method of claim 64, wherein the payload is one of a therapeutic agent, a virus or viral-like particle, and a liposome.

66. (canceled)

67. (canceled)

68. An isolated nucleic acid encoding the antibody as recited in claim 1.

69. A host cell comprising:
the nucleic acid of claim 68.

70. A cell line expressing the antibody of claim 1.

71. The cell line of claim 70, wherein:
the cell line is a hybridoma.

72. A method of producing an antibody of claim 1 in culture medium under conditions sufficient to produce the antibody.

73. A pharmaceutical composition comprising the antibody of any of claim 1, and a therapeutic agent and a pharmaceutically acceptable carrier.

74. The pharmaceutical composition of claim 73, wherein:
the therapeutic agent is one of the group consisting of: cytokines, cytotoxins, radionuclides, drugs, immunomodulators, therapeutic enzymes, anti-proliferative agents, and immunosuppressive agents.


SEQUENCE LISTING (88 sequences)

SEQ ID NO: 1 - protein, 121 aa, Artificial Sequence
  Description of Artificial Sequence Synthetic polypeptide
VTLKVCGPGILQPSQTLGLACTFSGISLSTSGMGLSWLRKPSGKALEWLASIWNNDNYYNPSLKSRLTISKETSNNQVFLKLTSVDTADSTTYFCAWRERTMVTTSMLWTTGVKEPQSPSP

SEQ ID NO: 2 - protein, 6 aa, Artificial Sequence
  Description of Artificial Sequence Synthetic peptide
TSGMGL

SEQ ID NO: 3 - protein, 16 aa, Artificial Sequence
  Description of Artificial Sequence Synthetic peptide
ASIWNNDNYYNPSLKS

SEQ ID NO: 4 - protein, 15 aa, Artificial Sequence
  Description of Artificial Sequence Synthetic peptide
AWRERTMVTTSMLWT

SEQ ID NO: 5 - protein, 108 aa, Artificial Sequence
  Description of Artificial Sequence Synthetic polypeptide
DILMTQSPASLSASVGENVTITCRASENIYSYLAWYQQKQGKSPQLLLYKEKTLAEGVSSRFSGSGSGTQFSLRINSLQPEDFGSYYCQHHYGIPWTFGGGTKLEIKR

SEQ ID NO: 6 - protein, 11 aa, Artificial Sequence
  Description of Artificial Sequence Synthetic peptide
RASENIYSYLA

SEQ ID NO: 7 - protein, 7 aa, Artificial Sequence
  Description of Artificial Sequence Synthetic peptide
KEKTLAE

SEQ ID NO: 8 - protein, 9 aa, Artificial Sequence
  Description of Artificial Sequence Synthetic peptide
QHHYGIPWT

SEQ ID NO: 9 - protein, 7 aa, Artificial Sequence
  Description of Artificial Sequence Synthetic peptide
GDVAFVK

SEQ ID NO: 10 - protein, 7 aa, Artificial Sequence
  Description of Artificial Sequence Synthetic peptide
NPDPWAK

SEQ ID NO: 11 - protein, 7 aa, Artificial Sequence
  Description of Artificial Sequence Synthetic peptide
NPDPWAK

SEQ ID NO: 12 - protein, 8 aa, Artificial Sequence
  Description of Artificial Sequence Synthetic peptide
DSAHGFLK

SEQ ID NO: 13 - protein, 8 aa, Artificial Sequence
  Description of Artificial Sequence Synthetic peptide
DSAHGFLK

SEQ ID NO: 14 - protein, 9 aa, Artificial Sequence
  Description of Artificial Sequence Synthetic peptide
APNHAVVTR

SEQ ID NO: 15 - protein, 10 aa, Artificial Sequence
  Description of Artificial Sequence Synthetic peptide
DGAGDVAFVK

SEQ ID NO: 16 - protein, 8 aa, Artificial Sequence
  Description of Artificial Sequence Synthetic peptide
YLFEEYVK

SEQ ID NO: 17 - protein, 8 aa, Artificial Sequence
  Description of Artificial Sequence Synthetic peptide
YLGEEYVK

SEQ ID NO: 18 - protein, 11 aa, Artificial Sequence
  Description of Artificial Sequence Synthetic peptide
HQTVPQNTGGK

SEQ ID NO: 19 - protein, 10 aa, Artificial Sequence
  Description of Artificial Sequence Synthetic peptide
DSGFQMNQLR

SEQ ID NO: 20 - protein, 10 aa, Artificial Sequence
  Description of Artificial Sequence Synthetic peptide
DSGFQMNQLR

SEQ ID NO: 21 - protein, 7 aa, Artificial Sequence
  Description of Artificial Sequence Synthetic peptide
GDVAFVK

SEQ ID NO: 22 - protein, 7 aa, Artificial Sequence
  Description of Artificial Sequence Synthetic peptide
NPDPWAK

SEQ ID NO: 23 - protein, 7 aa, Artificial Sequence
  Description of Artificial Sequence Synthetic peptide
NPDPWAK

SEQ ID NO: 24 - protein, 8 aa, Artificial Sequence
  Description of Artificial Sequence Synthetic peptide
DSAHGFLK

SEQ ID NO: 25 - protein, 8 aa, Artificial Sequence
  Description of Artificial Sequence Synthetic peptide
DSAHGFLK

SEQ ID NO: 26 - DNA, 368 bp, Artificial Sequence
  Description of Artificial Sequence Synthetic polynucleotide
caggttactctgaaagtgtgtggccctgggatattgcagccatcacagactctcggcctggcctgtactttctctgggatttcactgagtacttctggtatgggtttgagctggcttcgtaagccctcagggaaggctttagagtggctggcaagcatttggaataatgataattattacaacccatctttgaagagccggctcacaatctccaaggagacctccaacaaccaagtattccttaaactcaccagtgtggacactgcagattctaccacatacttctgtgcttggagagagcggactatggtaactacttctatgctatggactactggggtcaaggaacctcagtcaccgtctcctca

SEQ ID NO: 27 - DNA, 323 bp, Artificial Sequence
  Description of Artificial Sequence Synthetic polynucleotide
gacattctgatgacccagtctccagcctccctatctgcatctgtgggagaaaatgtcactatcacatgtcgagcaagtgaaaatatttacagttatttagcatggtatcagcaaaagcagggaaaatctcctcagctcctactctataaggaaaaaaccttagcagaaggtgtgtcatcaaggttcagtggcagtggatcaggcacacagttttctctgaggatcaacagcctgcagcctgaagattttgggagttattactgtcaacatcattatggtattccgtggacgttcggtggaggcaccaagctggaaatcaaacg

SEQ ID NO: 28 - protein, 15 aa, Homo sapiens
SASDLTWDNLKGKKS

SEQ ID NO: 29 - protein, 15 aa, Homo sapiens
AARRLYWDDLKRKLS

SEQ ID NO: 30 - protein, 10 aa, Homo sapiens
LDGTRKPVEE

SEQ ID NO: 31 - protein, 10 aa, Homo sapiens
LAGTESPVRE

SEQ ID NO: 32 - protein, 9 aa, Artificial Sequence
  Description of Artificial Sequence Synthetic peptide
KGDVAFVKH

SEQ ID NO: 33 - protein, 9 aa, Artificial Sequence
  Description of Artificial Sequence Synthetic peptide
KNPDPWAKN

SEQ ID NO: 34 - protein, 10 aa, Artificial Sequence
  Description of Artificial Sequence Synthetic peptide
KDSAHGFLKV

SEQ ID NO: 35 - protein, 11 aa, Artificial Sequence
  Description of Artificial Sequence Synthetic peptide
RAPNHAVVTRK

SEQ ID NO: 36 - protein, 12 aa, Artificial Sequence
  Description of Artificial Sequence Synthetic peptide
KDGAGDVAFVKH

SEQ ID NO: 37 - protein, 10 aa, Artificial Sequence
  Description of Artificial Sequence Synthetic peptide
KYLGEEYVKA

SEQ ID NO: 38 - protein, 13 aa, Artificial Sequence
  Description of Artificial Sequence Synthetic peptide
KHQTVPQNTGGKN

SEQ ID NO: 39 - protein, 12 aa, Artificial Sequence
  Description of Artificial Sequence Synthetic peptide
KDSGFQMNQLRG

SEQ ID NO: 40 - protein, 12 aa, Artificial Sequence
  Description of Artificial Sequence Synthetic peptide
KDSGFQMNQLRG

SEQ ID NO: 41 - protein, 13 aa, Artificial Sequence
  Description of Artificial Sequence Synthetic peptide
KSASDLTWDNLKG

SEQ ID NO: 42 - protein, 13 aa, Artificial Sequence
  Description of Artificial Sequence Synthetic peptide
KHSTIFENLANKA

SEQ ID NO: 43 - protein, 13 aa, Artificial Sequence
  Description of Artificial Sequence Synthetic peptide
KEFQLFSSPHGKD

SEQ ID NO: 44 - protein, 13 aa, Artificial Sequence
  Description of Artificial Sequence Synthetic peptide
KEGYYGYTGAFRC

SEQ ID NO: 45 - protein, 13 aa, Artificial Sequence
  Description of Artificial Sequence Synthetic peptide
KKDSGFQMNQLRG

SEQ ID NO: 46 - protein, 14 aa, Artificial Sequence
  Description of Artificial Sequence Synthetic peptide
KDSAHGFLKVPPRM

SEQ ID NO: 47 - protein, 14 aa, Artificial Sequence
  Description of Artificial Sequence Synthetic peptide
KKSASDLTWDNLKG

SEQ ID NO: 48 - protein, 15 aa, Artificial Sequence
  Description of Artificial Sequence Synthetic peptide
KCLKDGAGDVAFVKH

SEQ ID NO: 49 - protein, 14 aa, Artificial Sequence
  Description of Artificial Sequence Synthetic peptide
KMYLGYEYVTAIRN

SEQ ID NO: 50 - protein, 15 aa, Artificial Sequence
  Description of Artificial Sequence Synthetic peptide
KSKEFQLFSSPHGKD

SEQ ID NO: 51 - protein, 14 aa, Artificial Sequence
  Description of Artificial Sequence Synthetic peptide
KMYLGYEYVTAIRN

SEQ ID NO: 52 - protein, 16 aa, Artificial Sequence
  Description of Artificial Sequence Synthetic peptide
RTAGWNIPMGLLYNKI

SEQ ID NO: 53 - protein, 16 aa, Artificial Sequence
  Description of Artificial Sequence Synthetic peptide
RTAGWNIPMGLLYNKI

SEQ ID NO: 54 - protein, 16 aa, Artificial Sequence
  Description of Artificial Sequence Synthetic peptide
KEDPQTFYYAVAVVKK

SEQ ID NO: 55 - protein, 20 aa, Artificial Sequence
  Description of Artificial Sequence Synthetic peptide
KHQTVPQNTGGKNPDPWAKN

SEQ ID NO: 56 - protein, 23 aa, Artificial Sequence
  Description of Artificial Sequence Synthetic peptide
KIMNGEADAMSLDGGFVYIAGKC

SEQ ID NO: 57 - protein, 23 aa, Artificial Sequence
  Description of Artificial Sequence Synthetic peptide
KIMNGEADAMSLDGGFVYIAGKC

SEQ ID NO: 58 - protein, 23 aa, Artificial Sequence
  Description of Artificial Sequence Synthetic peptide
KIMNGEADAMSLDGGFVYIAGKC

SEQ ID NO: 59 - protein, 40 aa, Artificial Sequence
  Description of Artificial Sequence Synthetic polypeptide
RAIAANEADAVTLDAGLVYDAYLAPNNLKPVVAEFYGSKE

SEQ ID NO: 60 - protein, 99 aa, Artificial Sequence
  Description of Artificial Sequence Synthetic polypeptide
VTLKVSGPGILQPSQTLSLACTFSGISLSTSGMGLSWLRKPSGKALEWLASIWNNDNYYNPSLKSRLTISKETSNYQVFLKLTSVDTADSATYYGAWRE

SEQ ID NO: 61 - DNA, 297 bp, Artificial Sequence
  Description of Artificial Sequence Synthetic polynucleotide
gttactctgaaagtgtctggccctgggatattgcagccatcacagactctcagcctggcctgtactttctctgggatttcactgagtacttctggtatgggtttgagctggcttcgtaagccctcagggaaggctttagagtggctggcaagcatttggaataatgataactactacaacccatctttgaagagccggctcacaatctccaaggagacctccaactaccaagtattccttaaactcaccagtgtggacactgcagattctgccacatactacggtgcttggagagag

SEQ ID NO: 62 - DNA, 301 bp, Artificial Sequence
  Description of Artificial Sequence Synthetic polynucleotide
caggttactctgaaagagtctggccctggtatattgcagccctcccagaccctcagtctgacctgttctttctctgggttttcactgagcacttttggtatgggtgtgagctggattcgtcagccttcagggaagggtctggagtggctggcacacatttattgggatgatgacaagcactataacccatccttgaagagccggctcacaatctccaaggatacctccaacaaccaggtattcctcaagatcaccactgtggacactgcagatactgccacatactactgtgctcgaagag

SEQ ID NO: 63 - DNA, 294 bp, Artificial Sequence
  Description of Artificial Sequence Synthetic polynucleotide
caggttactctgaaagagtctggccctgggatattgcagccctcccagaccctcagtctgacttgttctttctctgggttttcactgagcacttctaatatgggtataggctggattcgtcagccttcagggaagggtctagagtggctggcacacatttggtggaatgatgataagtactataacccatccctgaagagccggctcacaatctccaaggatacctccaacaaccaggtattcctcaagatcaccagtgtggacactgcagatactgccacatactactgtgct

SEQ ID NO: 64 - DNA, 14 bp, Artificial Sequence
  Description of Artificial Sequence Synthetic oligonucleotide
actatggtaactac

SEQ ID NO: 65 - DNA, 14 bp, Artificial Sequence
  Description of Artificial Sequence Synthetic oligonucleotide
actatggtaactac

SEQ ID NO: 66 - DNA, 14 bp, Artificial Sequence
  Description of Artificial Sequence Synthetic oligonucleotide
actatggtgactac

SEQ ID NO: 67 - DNA, 49 bp, Artificial Sequence
  Description of Artificial Sequence Synthetic oligonucleotide
ctatgctatggactactggggtcaaggaacctcagtcaccgtctcctca

SEQ ID NO: 68 - DNA, 39 bp, Artificial Sequence
  Description of Artificial Sequence Synthetic oligonucleotide
gactactggggccaaggcaccactctcacagtctcctca

SEQ ID NO: 69 - DNA, 301 bp, Artificial Sequence
  Description of Artificial Sequence Synthetic polynucleotide
caggttactctgaaagagtctggccctggtatattgcagccctcgcagaccctcagtctgacttgttctttctctgggttttcactgagtacttttggtatgggtgtgagctggattcgtcagccttcagggaaggatctggagtggctggcacacatttattgggatgatgacaagcactataacccatccttgaagagccagctcagaatctccaaggatacctccaacaaccaggtattcctcaagatcaccactgtggacactgtagatactgccacatactactgtgctcgaagag

SEQ ID NO: 70 - DNA, 301 bp, Artificial Sequence
  Description of Artificial Sequence Synthetic polynucleotide
caggttactctgaaagagtctggccctggtatattgcagccctcccagaccctcagtctgacctgttctttctctgtgttttcactgagcacttttggtatgggtgtgagctggattcgtcagccttcagggaagggtctggagtggctggcacacatttattgggatgaggacaagcactataaaccatccttgaagagccggctcacaatctccaaggatacctccaacaaccaggtattcctcaagatcaccactgtggacactgcagatactgccacatactactctgctcgaagag

SEQ ID NO: 71 - DNA, 301 bp, Artificial Sequence
  Description of Artificial Sequence Synthetic polynucleotide
caggttactctgaaagagtctggccctgggatattgcagtcctcccagaccctcagtctgacttgttctttctctgggttttcactgagcacttctggtatgggtgtgagctggattcgtcagccttcaggaaagggtctggagtggctggcacacatttactgggatgatgacaagcgctataacccatccctgaagagccggctcacaatctccaaggatacctccagaaaccaggtattcctcaagatcaccagtgtggacactgcagatactgccacatactactgtgctcgaagag

SEQ ID NO: 72 - DNA, 285 bp, Artificial Sequence
  Description of Artificial Sequence Synthetic polynucleotide
caggttactctgaaagagtctggccctgggatattgcagccctcccagaccctcagtctgacttgttctttctctgggttttcactgagcacttctaatatgggtataggctggattcgtcagccttcagggaagggtctagagtggctggcacacatttggtggaatgatgataagtactataacccatccctgaagagccggctcacaatctccaaggatacctccaacaaccaggtattcctcaagatcaccactgtggacactgcagatactgccacatac

SEQ ID NO: 73 - DNA, 301 bp, Artificial Sequence
  Description of Artificial Sequence Synthetic polynucleotide
caggttactctgaaagagtctggccctgggatattgcagccctcccagaccctcagtctgacttgttctttctctgggttttcactgagcacttttggtatgggtgtaggctggattcgtcagccttcagggaagggtctggagtggctggcacacatttggtgggatgatgataagtactataacccagccctgaagagtcggctcacaatctccaaggatacctccaaaaaccaggtattcctcaagatcgccaatgtggacactgcagatactgccacatactactgtgctcgaatag

SEQ ID NO: 74 - DNA, 285 bp, Artificial Sequence
  Description of Artificial Sequence Synthetic polynucleotide
caggttactctgaaagagtctggccctggtatattgcagccctcccagaccctcagtctgacttgttctttctctgggttttcactgagcacttttggtatgggtataggctggattcgtcagccttcagggaagggtctagagtggctggcacacatttggtgggatgatgataagtactataacccagccctgaagagtcggctcacaatctccaaggatacctccaacaaccaggtattcctcaagatcaccagtgtggacactgcagatactgccacatac

SEQ ID NO: 75 - DNA, 301 bp, Artificial Sequence
  Description of Artificial Sequence Synthetic polynucleotide
cagattactcagaaagagtctggccctgggatattgcagccctcccagaccctcagtctgacttgttctttctctgggttttcactgagcacttctggtatgggtgtaggctggattcatcagccttcagggaatggtctggagtggctggcacacatttggtggaatgataataagtactataacacagccctgaagagccggctcacaatctccaaggatacctccaacaaccaggtattcctcaagatcgccagtgtggacactgcagatactgccacatactactgtgctcgaatag

SEQ ID NO: 76 - protein, 95 aa, Artificial Sequence
  Description of Artificial Sequence Synthetic polypeptide
DIQMTQSPASLSASVGETVTITCRASENIYSYLAWYQQKQGKSPQLLVYNAKTLAEGVPSRFSGSGSGTQFSLKINSLQPEDFGSYYCQHHYGTP

SEQ ID NO: 77 - DNA, 284 bp, Artificial Sequence
  Description of Artificial Sequence Synthetic polynucleotide
gacatccagatgactcagtctccagcctccctatctgcatctgtgggagaaactgtcaccatcacatgtcgagcaagtgagaatatttacagttatttagcatggtatcagcagaaacagggaaaatctcctcagctcctggtctataatgcaaaaaccttagcagaaggtgtgccatcaaggttcagtggcagtggatcaggcacacagttttctctgaagatcaacagcctgcagcctgaagattttgggagttattactgtcaacatcattatggtactcc

SEQ ID NO: 78 - DNA, 284 bp, Artificial Sequence
  Description of Artificial Sequence Synthetic polynucleotide
gacatccagatgactcagtctccagcctccctatctgcatctgtgggagaaactgtcaccatcacatgtcgagcaagtgggaatattcacaattatttagcatggtatcagcagaaacagggaaaatctcctcagctcctggtctataatgcaaaaaccttagcagatggtgtgccatcaaggttcagtggcagtggatcaggaacacaatattctctcaagatcaacagcctgcagcctgaagattttgggagttattactgtcaacatttttggagtactcc

SEQ ID NO: 79 - DNA, 284 bp, Artificial Sequence
  Description of Artificial Sequence Synthetic polynucleotide
gacatccagatgactcagtctccagcctccctatctgtatctgtgggagaaactgtcaccatcacatgtcgagcaagtgagaatatttacagtaatttagcatggtatcagcagaaacagggaaaatctcctcagctcctggtctatgctgcaacaaacttagcagatggtgtgccatcaaggttcagtggcagtggatcaggcacacagtattccctcaagatcaacagcctgcagtctgaagattttgggagttattactgtcaacatttttggggtactcc

SEQ ID NO: 80 - DNA, 38 bp, Artificial Sequence
  Description of Artificial Sequence Synthetic oligonucleotide
tggacgttcggtggaggcaccaagctggaaatcaaacg

SEQ ID NO: 81 - DNA, 35 bp, Artificial Sequence
  Description of Artificial Sequence Synthetic oligonucleotide
acgttcggaggggggaccaagctggaaataaaacg

SEQ ID NO: 82 - DNA, 284 bp, Artificial Sequence
  Description of Artificial Sequence Synthetic polynucleotide
gacattcagatgactcagtctccagcctccctatctgcatctgtgggagaaactgtcaccatcacatgtcaagcaagtgagaatattgccagtgatttagcatggtattagcagaaacagggaaaatctcctcagctcctggtctatgatgcgagaaacttagcagatggtgtgccatcaaggttcagtggcagtggatcaggcacacactattctctcaatatccacagcctgcagtctgaagatgttgcgagatattactgtcaacattattatggtactcc

SEQ ID NO: 83 - DNA, 284 bp, Artificial Sequence
  Description of Artificial Sequence Synthetic polynucleotide
acatccagatgactcagtctccagcctcctctttctgcatctctgggagaaagtgtcaccatcacatgtcaagcaagtgagaatattgacaattatttatcatggtatcagcaaaaaccaaggaaatctcctcagcccctgatcaattatacaaccaactttgcagatggggttccatcagggtctagtggcagtggatcaggcacacagttttctctcaagatcaacagcctgcaaccagaagatgttgcaagtcattactgtcaacatcattatagtactcc

SEQ ID NO: 84 - DNA, 284 bp, Artificial Sequence
  Description of Artificial Sequence Synthetic polynucleotide
gacatccagatgactcagtctccagcctccctggctgcatctgtgggagaaactgtcaccatcacatgtcgagcaagtgagaacatttactacagtttagcatggtatcagcagaagcaagggaaatctcctcagctcctgatctataatgcaaacagcttggaagatggtgtcccatcgaggttcagtggcagtggatctgggacacagtattctatgaagatcaacagcatgcagcctgaagataccgcaacttatttctgtaaacaggcttatgacgttcc

SEQ ID NO: 85 - DNA, 270 bp, Artificial Sequence
  Description of Artificial Sequence Synthetic polynucleotide
gacatccagatgactcagtctccagcttcactgtctgcatctgtgggagaaactgtcaccatcacatgtggagcaagtgagaatatttacggtgctttaaattggtatcagcggaaacagggaaaatctcctcagctcctgatctatggtgcaaccaacttggcagatggcatgtcatcgaggttcagtggcagtggatctggtagacagtattctctcaagatcagtagcctgcatcctgacgatgttgcaacgtattactgtcaaaat

SEQ ID NO: 86 - DNA, 285 bp, Artificial Sequence
  Description of Artificial Sequence Synthetic polynucleotide
gacatccagatgactcagtgtccagccacccctttctgcatctctggaagaaagtgtcaccatcacatgtcaagcaagtgagaatattgacaattatttatcatggtctcagcaaaaaccaaggaaatctcctcagcccctgatcaattatacgaccagcttggcagatggggttccatcaaggtctagtggcagtggatcaggcacacagttttctctcaagatcaacaacttgcaaacagaagatgttgcaagttactactgtcaacatcattattggactcc

SEQ ID NO: 87 - DNA, 284 bp, Artificial Sequence
  Description of Artificial Sequence Synthetic polynucleotide
gacattcagatgacccagtctcctgcctcccagtctgcatctctgggagaaagtgtcaccatcacatgcctggcaagtcagaccattggtacatggttagcatggtatcagcagaaaccagggaaatctcctcagctcctgatttatgctgcaaccagcttggcagatggggtcccatcaaggttcagtggtagtggatctggcacaaaattttctttcaagatcagcagcctacaggctgaagattttgtaagttattactgtcaacaactttacagtactcc

SEQ ID NO: 88 - DNA, 269 bp, Artificial Sequence
  Description of Artificial Sequence Synthetic polynucleotide
gacatccaggtgactcagtctccagcgtccctgtctgcacctgtgggagaaagtgtctccatcacatgtaaagcaagtgaagaaatttatagtgctttaaattggtatcagcagaaaccagggaaatctccacagctcctgatctattatgcaaccagcttgggagatgatgtgccctcaaggttcagtggcagtaaatctggcacacagtattccctcaagatcagcagcctgcagcctgaagatcttgcaacttattactgtgaaca